(19) United States
(12) Patent Application Publication
(10) Pub. No.: US 2026/0224512 A1
(43) Pub. Date: Aug. 6, 2026

(54) USE OF 5-METHOXY-2-AMINOINDAN ("MEAI") IN METHODS FOR TREATING METABOLIC SYNDROME

(71) Applicants: Clearmind Labs Corp., Vancouver (CA); Yissum Research Development Company of the Hebrew University of Jerusalem Ltd., Jerusalem (IL)

(72) Inventors: Adi ZULOFF-SHANI, Tel Aviv (IL); Joseph TAM, Jerusalem (IL); Saja BARAGHITHY, Jerusalem (IL)

(21) Appl. No.: 19/113,383

(22) PCT Filed: Sep. 20, 2023
(86) PCT No.: PCT/IB2023/059301

§ 371 Date: Mar. 19, 2025

Related U.S. Application Data
(60) Provisional application No. 63/408,683, filed on Sep. 21, 2022.

(51) Int. Cl. A61K 31/135; A61K 31/164; A61P 1/16; A61P 3/04; A61P 3/06; A61P 3/08

(52) U.S. Cl. CPC A61K 31/135; A61K 31/164; A61P 1/16; A61P 3/04; A61P 3/06; A61P 3/08

(57) ABSTRACT

The present disclosure relates to methods of treating metabolic conditions by administrating 5-methoxy-2-aminoindan, or a salt thereof, to a subject in need thereof in a therapeutically effective amount. The disclosure also relates to methods for treating metabolic conditions by administering 5-methoxy-2-aminoindan or a pharmaceutically acceptable salt thereof, and an N-acylethanolamine or a pharmaceutically acceptable salt thereof, to a subject in need thereof in a therapeutically effective amount.

### CROSS-REFERENCE TO RELATED APPLICATION

This application claims priority to U.S. Provisional Application No. 63/408,683, filed on Sep. 21, 2022, the contents of which are hereby incorporated by reference.

### FIELD OF THE DISCLOSURE

The present disclosure relates, inter alia, to methods for treating metabolic conditions and associated syndromes by administering a therapeutically effective amount of 5-methoxy-2-aminoindan (“MEAI”). In certain embodiments, the methods of treatment comprise administering MEAI in combination with one or more N-acylethanolamines, for example, palmitoylethanolamide (“PEA”).

### BACKGROUND OF THE DISCLOSURE

Obesity is a chronic disease reaching epidemic proportions, with more than one-third (34.9% or 78.6 million) of U.S. adults considered obese. Obesity has been described as a catalyst for a number of conditions, most notably cardiovascular disease, type 2 diabetes mellitus (T2DM) and non-alcoholic fatty liver disease (NAFLD). While several metabolic factors have been linked to the development of obesity, the exact molecular mechanisms involved are not fully understood.

Moreover, despite the severity of obesity and its comorbidities there are a few anti-obesity agents available on the market. The etiologies of obesity have been attributed to eating behavior or fast food, personality issues, depression, or genetics. Food addiction is currently one of the emerging hypotheses for the epidemic spread of obesity, which is often associated with both substance-related disorders and eating disorders (most prominently binge-eating). There is evidence that bingeing on sugar-dense, palatable foods increases extracellular dopamine in the striatum and thereby possesses an addictive potential. Moreover, there appear to be several biological and psychological similarities between food addiction and drug dependence including craving and loss of control.

Nevertheless, as shown by the limited options for treating obesity and other metabolic disorders, a need remains for improved treatment options.

### SUMMARY OF THE DISCLOSURE

In some embodiments, provided herein is a method for treating a metabolic condition, e.g., one associated with one or more metabolic syndrome comprising administering to a subject in need thereof a therapeutically acceptable amount of a pharmaceutical composition comprising 5-methoxy-2-aminoindan or a pharmaceutically acceptable salt thereof, thereby treating the metabolic condition. In certain embodiments, the 5-methoxy-2-aminoindan or pharmaceutically acceptable salt thereof is administered as a dose of about 20 to about 520 mg. In other embodiments, the 5-methoxy-2-aminoindan or pharmaceutically acceptable salt thereof is administered as a dose of about 0.5 to about 40 mg.

In some embodiments, the 5-methoxy-2-aminoindan or pharmaceutically acceptable salt thereof is administered as a dose of about 20 to about 100 mg, about 25 to about 90 mg, about 30 to about 80 mg, about 40 to about 70 mg, or about 50 to about 60 mg. In other embodiments, the dose is administered in a single dose or as more than one divided dose. In other embodiments, the dose is administered daily in a single dose or as more than one divided dose. In certain embodiments, the 5-methoxy-2-aminoindan or a pharmaceutically acceptable salt thereof is administered twice a day.

In some embodiments, the therapeutically effective amount of 5-methoxy-2-aminoindan or a pharmaceutically acceptable salt thereof comprises about 0.0084 to about 0.67 mg/kg body weight/day, about 0.33 to about 8.67 mg/kg body weight/day, about 0.33 to about 1.67 mg/kg body weight/day, about 0.42 to about 1.5 mg/kg body weight/day, about 0.5 to about 1.33 mg/kg body weight/day, about 0.67 to about 1.17 mg/kg body weight/day, or about 0.83 to about 1.0 mg/kg body weight/day.

In some embodiments, the administered pharmaceutical composition further comprises at least one pharmaceutically acceptable carrier and/or excipient. In particular embodiments, the pharmaceutical composition is a free-flowing powder, a tablet, a capsule, a lozenge, a liquid, a liquid concentrate, suspension, or a syrup. In certain embodiments, the pharmaceutical composition is a unit dosage form composition. In some embodiments, an amount of 5-methoxy-2-aminoindan or pharmaceutically acceptable salt thereof in the unit dosage form is about 20 to about 520 mg, about 0.5 to about 40 mg, about 20 to about 100 mg, about 25 to about 90 mg, about 30 to about 80 mg, about 40 to about 70 mg, or about 50 to about 60 mg. In particular embodiments, the amount of 5-methoxy-2-aminoindan or pharmaceutically acceptable salt thereof is about 50 mg.

In some embodiments, administration of the pharmaceutical composition is oral, sublingual, buccal, vaginal, rectal, parenteral, transdermal, or by inhalation. In certain embodiments, the parenteral administration is intravenous, intramuscular, or subcutaneous.

In some embodiments, treating a metabolic condition, e.g., one associated with one or more metabolic syndrome, comprises administering a therapeutically acceptable amount of a pharmaceutical composition comprising 5-methoxy-2-aminoindan or a pharmaceutically acceptable salt thereof, and separately, concurrently, or simultaneously administering an N-acylethanolamine or a pharmaceutically acceptable salt thereof, thereby treating the metabolic disorder. The N-acylethanolamine or a pharmaceutically acceptable salt thereof may be in the same or a separate pharmaceutical composition as the 5-methoxy-2-aminoindan or a pharmaceutically acceptable salt thereof.

In some embodiments, the N-acylethanolamine or pharmaceutically acceptable salt thereof is administered as a dose of about 200 to about 1800 mg. In some embodiments, the N-acylethanolamine or pharmaceutically acceptable salt thereof is administered as a dose of about 250 to about 1550, about 300 to about 1200 mg, about 350 to about 950 mg, about 400 to about 700 mg, about 450 to about 600 mg, or about 500 to about 550 mg. In other embodiments, the dose is administered in a single dose or as more than one divided dose. In other embodiments, the dose is administered daily as a single dose or as more than one divided dose. In certain embodiments, the N-acylethanolamine or a pharmaceutically acceptable salt thereof is administered twice a day.

In some embodiments, the 5-methoxy-2-aminoindan and N-acylethanolamine may be administered at a molar ratio ranging from about 1:0.2 to about 1:2000.

In some embodiments, the N-acylethanolamine is selected from the group consisting of N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), salts thereof and any combination thereof. Each possibility represents a separate embodiment of the present disclosure. In certain embodiments, the N-acylethanolamine is PEA or a salt thereof. In certain embodiments, the N-acylethanolamine consists of PEA or a salt thereof. In certain embodiments, the N-acylethanolamine consists of PEA.

In some embodiments, the therapeutically effective amount of N-acylethanolamine or a pharmaceutically acceptable salt thereof comprises about 2.5 to about 36.0 mg/kg body weight/day, about 3.12 to about 31.0 mg/kg body weight/day, about 3.75 to about 24.0 mg/kg body weight/day, about 4.38 to about 19.0 mg/kg body weight/day, about 5.0 to about 14.0 mg/kg body weight/day, about 5.62 to about 12.0 mg/kg body weight/day, or about 6.25 to about 11.0 mg/kg body weight/day.

In some embodiments, the pharmaceutical composition further comprises at least one pharmaceutically acceptable carrier and/or excipient. In particular embodiments, the pharmaceutical composition is a free-flowing powder, a tablet, a capsule, a lozenge, a liquid, a liquid concentrate, suspension, or a syrup. In certain embodiments, the pharmaceutical composition is a unit dosage form composition.

In some embodiments, administration of the pharmaceutical composition is oral, sublingual, buccal, vaginal, rectal, parenteral, transdermal, or by inhalation. In certain embodiments, the parenteral administration is intravenous, intramuscular, or subcutaneous.

In some embodiments, administration is oral, mucosal, nasal, sublingual, inhalational, topical, rectal, vaginal, or parenteral route. In certain embodiments, parenteral administration is intravenous, intramuscular, or subcutaneous.

In some embodiments, treating a metabolic condition comprises reducing one or more metabolic syndrome, e.g., one or more of decreasing blood pressure, decreasing blood sugar, reducing body fat around the waist, normalizing abnormal cholesterol or triglyceride levels, reducing obesity, reducing overweight, reducing body weight, increasing lean mass, reducing fat mass, reducing adiposity, increasing energy expenditure, improving glycemic control, decreasing hepatic steatosis, decreasing sugar intake, decreasing food intake, maintaining glucose homeostasis, lowering dyslipidemia, or preserving liver function. In certain embodiments, improving glycemic control involves one or more of improving glucose metabolism, reducing fasting blood glucose level, or reducing insulin level. In some embodiments, increasing energy expenditure involves one or more of increasing oxygen consumption and carbon dioxide emission, increasing fat oxidation, or increasing locomotive activity.

In some embodiments, the improved metabolic syndrome involves reducing obesity. In some embodiments, treating metabolic syndrome involves reducing overweight associated with obesity. In some embodiments, treating metabolic syndrome preserves lean mass of a subject. In some embodiments, treating metabolic syndrome decreases fat mass of a subject. In some embodiments, treating metabolic syndrome reduces adiposity of a subject.

In some embodiments, the improved metabolic syndrome comprises increasing energy expenditure. In some embodiments, treating metabolic syndrome increases energy expenditure and food consumption is unchanged. In some embodiments, treating metabolic syndrome increases energy expenditure and fat utilization is increased. In some embodiments, treating metabolic syndrome increases energy expenditure and locomotive activity is normalized without over-stimulatory effects.

In some embodiments, the improved metabolic syndrome comprises improving glycemic control. In some embodiments, treating metabolic syndrome reverses hyperglycemia, glucose intolerance, or hyperinsulinemia. In some embodiments, treating metabolic syndrome treats hepatic steatosis. In some embodiments, treating hepatic steatosis involves one or more of reducing hepatic lipid accumulation, hepatic triglyceride levels, or hepatic cholesterol levels. In some embodiments, treating metabolic syndrome preserves glucose homeostasis. In some embodiments, preserving glucose homeostasis involves one or more of enhancing glucose tolerance, attenuating insulin resistance, reducing dyslipidemia, or reducing hepatic lipid accumulation.

In some embodiments, a pharmaceutical composition comprising 5-methoxy-2-aminoindan or a pharmaceutically acceptable salt thereof is used for treating a metabolic syndrome according to any of the preceding embodiments.

In some embodiments, a pharmaceutical composition comprising 5-methoxy-2-aminoindan or a pharmaceutically acceptable salt thereof and palmitoylethanolamide or a pharmaceutically acceptable salt thereof is used for treating a metabolic syndrome according to any of the preceding embodiments.

### BRIEF DESCRIPTION OF THE FIGURES

The foregoing summary, as well as the following detailed description of the disclosure, will be better understood when read in conjunction with the appended drawings. For the purpose of illustrating the present disclosure, the attached drawings illustrate some, but not all, alternative embodiments. It should be understood, however, that the disclosure is not limited to the precise arrangements and instrumentalities shown. These figures, which are incorporated into and constitute part of the specification, assist in explaining the principles of the disclosures.

FIG. 1 shows acute effects of MEAI administration on food intake patterns and energy utilization. Shown are experimental design (FIG. 1A), cumulative food intake and sum of food intake (FIG. 1B,C), cumulative water intake and sum of water intake (FIG. 1D, E), respiratory exchange rate (RER) (FIG. 1F), rate of oxygen consumption (FIG. 1G), rate of carbon dioxide emission (FIG. 1H), total energy expenditure (TEE) (FIG. 1I,J), fat oxidation (FIG. 1K), and carbohydrate oxidation (FIG. 1L). Data represent mean±SEM from 6-8 mice per group. *P<0.05 relative to Vehicle-treated group.

FIG. 2 shows acute alterations in the activity profile following MEAI administration. Shown are total ambulatory activity (FIG. 2A), pedestrian locomotion (FIG. 2B), pedestrian speed (FIG. 2C), wheel running distance (FIG. 2D), and total pedometer count (FIG. 2E). Data represent mean±SEM from 6-8 mice per group. *P<0.05 relative to Vehicle-treated group.

FIG. 3 shows the acute effects of MEAI on sweet tase preference. Shown are sucrose preference percentages compared to sterile water over a test period of 48 hours. Data represent mean±SEM from 8 mice per group. *P<0.05 relative to Vehicle-treated group.

FIG. 4 shows the chronic administration of MEAI attenuates weight gain and body composition changes associated with obesity. Shown are the experimental design to test efficacy of MEAI in a HFD-induced obesity model (FIG. 4A), time course changes of body weight (FIG. 4B), total body weight at the end of the experiment (FIG. 4C), total body weight change at the end of the experiment (FIG. 4D), lean mass percentage of the overall body weight (FIG. 4E), lean mass in grams (FIG. 4F), fat mass percentage of overall body weight (FIG. 4G), and fat mass in grams (FIG. 4H). Data represent mean±SEM from 8-11 mice per group. *P<0.05 relative to STD vehicle; #P<0.05 relative to HFD vehicle.

FIG. 5 shows the effect of chronic MEAI administration on food consumption and energy metabolism. Shown are hourly food consumption rate (FIG. 5A), cumulative food consumption, sum of food consumption in grams/day, sum of food consumption in kcal/day (FIG. 5B,C,D), cumulative water intake (FIG. 5E), respiratory exchange rate (RER) (FIG. 5F), rate of oxygen consumption (FIG. 5G), rate of carbon dioxide emission (FIG. 5H), total energy expenditure rate (FIG. 5I,J), fat oxidation (FIG. 5K), and carbohydrate oxidation (FIG. 5L). Data represent mean±SEM from 8-11 mice per group. *P<0.05 relative to STD vehicle; #P<0.05 relative to HFD vehicle.

FIG. 6 shows locomotive activity following chronic MEAI administration in HFD-induced obesity. Shown are 24-hour time course changes of ambulatory activity (FIG. 6A), pedestrian locomotion (FIG. 6B), pedestrian locomotion speed (FIG. 6C), total distance traveled (FIG. 6D), wheel running distance (FIG. 6E), wheel speed (FIG. 6F), and a chart indicating percentages of time spent by mice engaging in various activities (FIG. 6G). Data represent mean±SEM from 8-11 mice per group. *P<0.05 relative to STD vehicle; #P<0.05 relative to HFD vehicle.

FIG. 7 shows effects of chronic MEAI administration on glucose tolerance and insulin sensitivity. Shown are blood glucose levels in a glucose tolerance test (FIG. 7A), area-under-curve (AUC) values of the glucose tolerance test (FIG. 7B), blood glucose percentages in an insulin tolerance test (FIG. 7C), AUC values of the insulin tolerance test (FIG. 7D), fasting blood glucose levels (FIG. 7E), serum insulin levels (FIG. 7F), homeostasis model assessment insulin resistance (HOMA-IR) values (FIG. 7G), and insulin sensitivity index (ISI) values (FIG. 7H). Data represent mean±SEM from 8-11 mice per group. *P<0.05 relative to STD vehicle; #P<0.05 relative to HFD vehicle.

FIG. 8 shows a circulating lipids profile following MEAI treatment. Shown are HDL levels (FIG. 8A), LDL levels (FIG. 8B), HDL-to-LDL ratio (FIG. 8C), cholesterol levels (FIG. 8D), and triglyceride levels (FIG. 8E). Data represent mean±SEM from 8-11 mice per group. *P<0.05 relative to STD vehicle; #P<0.05 relative to HFD vehicle.

FIG. 9 shows the effects of MEAI on kidney weight and function. Shown are Kidney weight (FIG. 9A), Kidney weight to body weight ratio (FIG. 9B), and BUN as measured by the COBAS Chemistry analyzer (FIG. 9C). Data represent mean±SEM from 8-11 mice per group. *P<0.05 relative to STD vehicle; #P<0.05 relative to HFD vehicle.

FIG. 10 shows that MEAI improves obesity-associated liver steatosis. Shown are weight of liver samples after chronic treatment with MEAI compared to vehicle (FIG. 10A), liver weight to body weight ratio (FIG. 10B), ALT levels (FIG. 10C), AST levels (FIG. 10D), ALP levels (FIG. 10E), hepatic triglyceride content (FIG. 10F), hepatic cholesterol content (FIG. 10G), Oil Red O staining area percentage (FIG. 10H), and Oil Red O stained samples demonstrating lipid vacuoles in hepatocytes (FIG. 10I). Data represent mean±SEM from 8-11 mice per group. *P<0.05 relative to STD vehicle; #P<0.05 relative to HFD vehicle.

### DETAILED DESCRIPTION OF THE DISCLOSURE

Compounds derived from 2-aminoindan may be used in the methods disclosed herein. Such compounds have been shown to selectively bind to the dopamine D3 receptor. U.S. Pat. No. 5,708,018 discloses some 2-aminoindan derivatives and hypothesizes that these 2-aminoindan derivatives may be useful in treating CNS disorders associated with dopamine D3 receptor. One such compound is 5-methoxy-2-aminoindan (“MEAI”), which the chemical formula is:

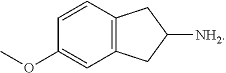

Other 2-aminoindan derivatives that may be used in the disclosure herein may be represented by a compound of Formula I:

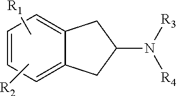

Wherein each of R1 and R2 is independently selected from the group consisting of H, (C1-C8)alkyl, (C2-C8)alkenyl, (C2-C8)alkynyl, (C3-C8) cycloalkyl, aryl, heteroaryl, heteroalicyclic, —O(C1-C8)alky, OH, —OSO2CF3, —OSO2-(C1-C8)alkyl, —SOR5, —CO2R5, —CONR5R6, —COR5, —CF3, CN, —SR5, —SO2NR5R6, —SO2R5, —OCO—(C1-C8)alkyl, —NCO—(C1-C8)alkyl, —CH2O—(C1-C8)alkyl, —(C1-C6)alkyl-OH, —NHSO2R5, and halogen, or, alternatively, R1 and R2 together with two or more of the phenyl carbon atoms form a —X1-(CR5R6)m-X2-ring, wherein each of X1 and X2 is independently selected from C, O, NH or S and m is 1, 2, 3, or 4;
each of R3 and R4 is independently selected from the group consisting of H, (C1-C8)alkyl, (C2-C8)alkenyl, (C2-C8)alkynyl, (C3-C8) cycloalkyl, and —(CH2) p-thienyl, wherein p is 1, 2, 3, or 4, or alternatively, R3 and R4 are joined together to form a heterocyclic ring (heteroalicyclic or heteroaryl) containing the nitrogen atom to which they are attached; and
each of R5 and R6 is independently selected from the group consisting of H, (C1-C8)alkyl, (C2-C8)alkenyl, (C3-C8) cycloalkyl and aryl.

In some embodiments, the 2-aminoindan derivative represented by Formula I as presented herein, is defined as follows:
each of R1 and R2 is independently selected from the group consisting of H, (C1-C8)alkyl, (C2-C8)alkenyl, (C2-C8)alkynyl, (C3-C8) cycloalkyl, aryl, —OCH3, OH, —OSO2CF3, —OSO2CH3, —SOR5, —CO2R5, —CONR5R6-COR5, —CF3, —CN, —SR5, —SO2NR5R6, —SO2R5, —CH2-OH, halogen, phthalimidyl, thiophenyl, pyrrolyl, pyrrolinyl, oxazolyl, or, alternatively, R1 and R2 together with two or more of the phenyl carbon atoms form a —O(CH2)mO- ring, wherein m is 1 or 2;
R3 and R4 are joined together to form a heterocyclic ring containing 4 to 8 carbon atoms with the nitrogen atom to which they are attached; and
each of R5 and R6 is independently selected from the group consisting of H, (C1-C8)alkyl, (C2-C8)alkenyl, and (C3-C8) cycloalkyl.

Non-limiting examples of 2-aminoindane derivatives include:
(1) 5-methoxy-2-aminoindan;
(2) 5,6-dimethoxy-2-aminoindan:
(3) 5-methoxy-2-(N-propylamino)indan;
(4) 5,6-dimethoxy-2-(N-propylamino)indan;
(5) 5,6-dimethoxy-2-(di-N-butylamino)indan;
(6) 5-(trifluoromethylsulfonyloxy)-6-hydroxy-2-(di-N-propylamino)indan;
(7) 5-(trifluoromethylsulfonyloxy)-2-(N-propylamino)indan;
(8) 5,6-(di-trifluoromethylsulfonyloxy)-2-(N-propylamino)indan;
(9) 5,6-dimethoxy-2 (pyrrolidino)indan;
(10) 5-(trifluoromethylsulfonyloxy)-6-acetoxy-2-(di-N-propylamino)indan;
(11) 5-trifluromethansulfonyloxy-6-methoxy-2-(di-N-propylamino)indan;
(12) 5,6-ethylenedioxy-2-(di-N-propylamino)indan;
(13) 5,6-methylenedioxy-2-(di-N-propylamino)indan;
(14) 5-hydroxy-2-(n-propylamino)indan;
(15) 5,6-dihydroxy-2-(n-propylamino)indan;
(16) 4-methyl-2-aminoindan;
(17) 4,5-di-methyl-2-aminoindan;
(18) 5,6-di-methyl-2-amninoindan;
(19) 6-methyl-2-aminoindan;
(20) 4-fluoro-2-aminoindan;
(21) 5-(i-propyl)-2-aminoindan;
(22) 4,6-dimethyl-2-aminoindan;
(23) 4,7-dimethyl-2-aminoindan;
(24) 5-(t-butyl)-2-aminoindan:
(25) 5-propyl-2-aminoindan;
(26) 5-fluoro-2-(di-N-propylamino)indan;
(27) 6-methylenedioxy-2-(di-N-propylamino)indan:
(28) 5,6-dimethoxy-2 (pyrrolidino)indan;
(29) 5,6-(di-carbomethoxy)-2-(di-N-propylamino)indan;
(30) 5-(carbomethoxy)-6-hydroxy-2-(di-N-propylamino)indan;
(31) 5-bromo-2-(dipropylamino)indan;
(32) (6-methylsulfanyl-indan-2-yl)-dipropyl-amine;
(33) (6-methylsulfonyl-indan-2-yl)-dipropyl-amine;
(34) (6-methylsulfinyl-indan-2-yl)-dipropyl-amine;
(35) 2-dipropylamino-indan-5-carbaldehyde;
(36) (5-iodo-indan-2-yl)-dipropyl-amine;
(37) (4-iodo-indan-2-yl)-dipropyl-amine;
(38) toluene-4-sulfonic acid 2-dipropylamino-indan-5-yl ester;
(39) toluene-4-sulfonic acid 2-dipropylamino-6-hydroxy-indan-5-yl ester;
(40)N-[2-(benzyl-propylamino)-indan-5-yl]-4-methyl benzene-sulfonamide;
(41)N-[2-(benzyl-propyl-amino)-indan-5-yl]methanesulfonamide;
(42) 2-[2-(benzyl-propyl-amino)-indan-5-yl]-isoindole-1,3-dione;
(43) benzyl-propyl-(6-pyrrol-1-yl-indan-2-yl)-amine;
(44) propyl-(6-pyrrol-1-yl-indan-2-yl)-amine;
(45) propyl-(6-pyrrolidin-1-yl-indan-2-yl)-amine;
(46) dipropyl-(6-pyrrolidin-1-yl-indan-2-yl)-amine;
(47) cyclopropanecarboxylic acid-[2-(benzyl-propyl-amino)-indan-5-yl]acetamide;
(48)N-[2-(benzyl-propyl-amino)-indan-5-yl]propionamide;
(49)N-[2-(benzyl-propyl-amino)-indan-5-yl]-2,2-dimethyl propionamide;
(50) 5-(2-propenyloxy)-2-(di-N-propylamino)-indan;
(51) 5,6 di-toluenesulfonyloxy-2-(di-N-propylamino)indan;
(52) 5-methanesulfonyloxy-2-(di-N-propylamino)indan;
(53) 5-carbomethoxy-2-(di-N-propylamino)indan;
(54) 5-carboxamido-2-(di-N-propylamino)indan;
(55) 5,6-di-trifluoromethansulfonyloxy-2-(propylamino)indan;
(56) 4-methyl-2-(di-N-propylamino)indan;
(57) 4,5-di-methyl-2-(di-N-propylamino)indan;
(58) 5,6-di-methyl-2-(di-N-propylamino)indan;
(59) 5-methyl-2-(di-N-propylamino)indan;
(60) 4-fluoro-2-(N-propyl)aminoindan;
(61) 5-(i-propyl)-2-(di-N-propylamino)indan;
(62) 5-(i-propyl)-2-(N-propylamino)indan;
(63) 4,6-dimethyl-2-(di-N-propylamino)indan;
(64) 4,7-dimethyl-2-(di-N-propylamino)indan;
(65) 5-propyl-2-(di-N-propylamino)indan;
(66) 5-(t-butyl)-2-(dim-propylamino)indan;
(67) 5-trifluoromethyl-2-(di-N-propylamino)indan;
(68) 5-sulfoxamido-2-(di-N-propylamino)indan;
(69) 5-(3-thiophene)-2-(di-N-propylamino)indan;
(70) 5-ethynyl-2-(di-N-propylamino)indan;
(71) 5-acetyl-2-(di-N-propylamino)indan;
(72) 5-cyano-2-(di-N-propylamino)indan;
(73) 5-carbomethoxy-6-acetoxy-2-(di-N-propylamino)indan;
(74) 5-carbomethoxy-6-trifluoromethanesulfonyloxy-2-(di-N-propylamino)indan;
(75) 5-carbomethoxy-6-methoxy-2-(di-N-propylamino)indan;
(76) 5-formyl-6-methoxy-2-(di-N-propylamino)indan:
(77) 5-hydroxymethyl-6-methoxy-2-(di-N-propylamino)indan;
(78) 5-carboxy-6-methoxy-2-(di-N-propylamino)indan;
(79) 5-acetyl-6-methoxy-2-(di-N-propylamino)indan;
(80) 5-carboxamido-6-methoxy-2-(di-N-propylamino)indan;
(81) 5-ethynyl-6-methoxy-2-(di-N-propylamino)indan:
(82) 5-cyano-6-methoxy-2-(di-N-propylamino)indan; and
(83) 5,6-di-(hydroxymethyl-2-(di-N-propylamino)indan.

In some embodiments described herein, the 2-aminoindan derivative represented by Formula I is any one of the compounds (1)-(13) above, in which the phenyl moiety is substituted by one or two —OCH3, or —OSO2CF3 groups, or the phenyl moiety bears a —O(CH2)mO- ring, wherein m is 1 or 2, fused thereto. The structural formulas of compounds 1-13 are depicted in Table A hereinunder.

TABLE A

| Compound | | |
|---|---|---|
| No. | Name | Structure |
| 1 | 5-methoxy-2-aminoindan | 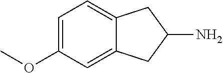 |
| 2 | 5,6-dimethoxy-2- aminoindan | 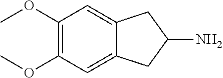 |
| 3 | 5-methoxy-2-(N- propylamino)indan | 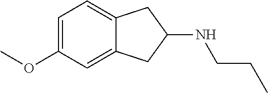 |
| 4 | 5,6-dimethoxy-2-(N- propylamino)indan | 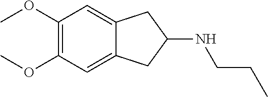 [SMILES: CCCNC1Cc2cc(OC)c(OC)cc2C1] |
| 5 | 5,6-dimethoxy-2-(di-N- butylamino)indan | 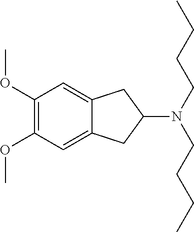 [SMILES: CCCCN(CCCC)C1Cc2cc(OC)c(OC)cc2C1] |
| 6 | 5-(trifluoromethyl- sulfonyl)-6-hydroxy-2- (di-N-propylamino)indan | 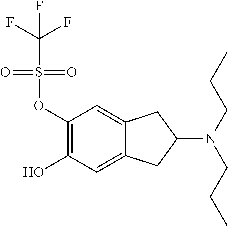 [SMILES: CCCN(CCC)C1Cc2cc(O)c(OS(=O)(=O)C(F)(F)F)cc2C1] |
| 7 | 5-(trifluoromethyl- sulfonyloxy)-2-(N- propylamino)indan | 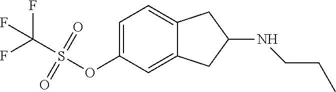 [SMILES: CCCNC1Cc2ccc(OS(=O)(=O)C(F)(F)F)cc2C1] |
| 8 | 5,6-(di-trifluoromethyl- sulfonyloxy)-2-(N- propylamino)indan | 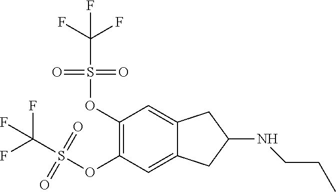 [SMILES: CCCNC1Cc2cc(OS(=O)(=O)C(F)(F)F)c(OS(=O)(=O)C(F)(F)F)cc2C1] |
| 9 | 5,6-dimethoxy-2- (pyrrolidino)indan | 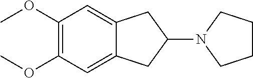 [SMILES: COc1cc2c(cc1OC)CC(N1CCCC1)C2] |
| 10 | 5- (trifluoromethylsulfonyloxy)- 6-acetoxy-2-(di-N- propylamino)indan | 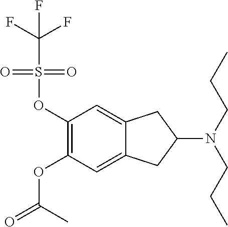 [SMILES: CCCN(CCC)C1Cc2cc(OC(C)=O)c(OS(=O)(=O)C(F)(F)F)cc2C1] |
| 11 | 5- trifluoromethanesulfonyloxy- 6-methoxy-2-(di-N- propylamino)indan | 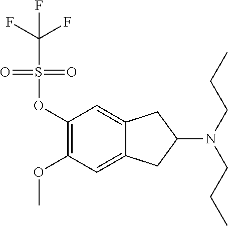 [SMILES: CCCN(CCC)C1Cc2cc(OC)c(OS(=O)(=O)C(F)(F)F)cc2C1] |
| 12 | 5,6-ethylenedioxy-2-(di-N- propylamino)indan | 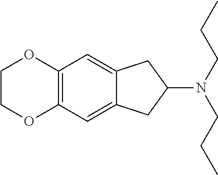 [SMILES: CCCN(CCC)C1Cc2cc3c(cc2C1)OCCO3] |
| 13 | 5,6-methylenedioxy-2-(di- N-propylamino)indan | 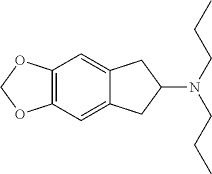 [SMILES: CCCN(CCC)C1Cc2cc3c(cc2C1)OCO3] |

N-acylethanolamines (NAEs) are a type of fatty acid amide, lipid-derived signaling molecules. They are formed when one of several types of acyl groups is linked to the nitrogen atom of ethanolamine. These amides conceptually can be formed from a fatty acid and ethanolamine with the release of a molecule of water, but the known biological synthesis uses a specific phospholipase D to cleave the phospholipid unit from N-acylphosphatidylethanolamines. Examples of N-acylethanolamines include anandamide (the amide of arachidonic acid (20:4 omega-6) and ethanolamine), N-Palmitoylethanolamine (the amide of palmitic acid (16:0) and ethanolamine), N-Oleoylethanolamine (the amide of oleic acid (18:1) and ethanolamine), N-Stearoylethanolamine (the amide of stearic acid (18:0) and ethanolamine) and N-Docosahexaenoylethanolamine (the amide of docosahexaenoic acid (22:6) and ethanolamine).

Palmitoylethanolamide (PEA, also known as N-(2-hydroxyethyl) hexadecanamide; Hydroxyethylpalmitamide; palmidrol; N-palmitoylethanolamine; and palmitylethanolamide) is an example NAE and is an endogenous fatty acid amide, belonging to the class of nuclear factor agonists. The chemical structure of PEA is:

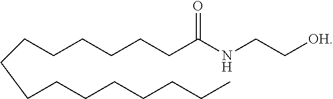 [SMILES: CCCCCCCCCCCCCCCC(=O)NCCO]

PEA has been demonstrated to bind to a receptor in the cell nucleus (a nuclear receptor) and exerts a variety of biological functions related to chronic pain and inflammation. Studies have shown that PEA interacts with distinct non-CB1/CB2 receptors, suggesting that PEA utilizes a unique “parallel” endocannabinoid signaling system. This concept was further supported by growing evidence that PEA production and inactivation can occur independently of AEA and 2-AG production and inactivation. Much of the biological effects of PEA on cells can be attributed to its affinity to PPAR (particularly PPAR-.alpha, and PPAR-.gamma.). PEA was shown to have an affinity to cannabinoid-like G-coupled receptors GPR55 and GPR119 as well as the transient receptor potential vanilloid type 1 receptor (TRPV1). PEA has been shown to have anti-inflammatory, anti-nociceptive, neuro-protective, and anti-convulsant properties.

In various embodiments, disclosed herein are methods of treating metabolic conditions, e.g., by reducing one or more metabolic syndrome as a whole and more particularly, but not exclusively, to methods for treating obesity, comprising administering to a subject in need thereof a therapeutically effective amount of MEAI, or a pharmaceutically acceptable salt thereof. In some embodiments, the method further comprises administering an N-acylethanolamine, or a pharmaceutically acceptable salt thereof.

In some embodiments, the disclosure also provides preclinical evidence for the efficacy of MEAI in regulating energy metabolism and mitigating obesity and its related metabolic abnormalities. In some embodiments, MEAI demonstrates remarkable effectiveness in preventing or alleviating various conditions associated with a metabolic syndrome. In addition to mitigating adiposity and reducing body weight, MEAI may also maintain glucose homeostasis, lower dyslipidemia, and preserve liver function, possibly improve fat utilization and oxidation. In some embodiments, MEAI may have potential as a novel therapeutic option for obesity and its related metabolic disorders.

### Definitions

“Metabolic Syndrome” refers to a cluster of symptoms (which may occur together), which may increase a subject's risk of having or which are associated with a metabolic condition such as obesity, heart disease, stroke, or type 2 diabetes. These symptoms, alone or in combination, include increased blood pressure, high blood sugar, and/or insulin resistance (type-2 diabetes), excess body fat around the waist, and abnormal cholesterol (low HDL and/or high LDL) triglyceride levels with or without fatty liver disease, obesity, overweight, excess body weight, excess fat mass, excess adiposity, decreased energy expenditure, abnormal glycemic control, increased hepatic steatosis, excess sugar intake, excess food intake or consumption, abnormal glucose homeostasis, increased dyslipidemia, or abnormal liver function. In some circumstances, a person exhibiting a metabolic syndrome may have excess body fat around the waist and/or be obese.

“Metabolic condition” refers to a group of conditions associated with one or more metabolic syndrome. Examples of metabolic conditions include obesity, diabetes, diabetes associated with obesity, cardiovascular disease, nonalcoholic steatohepatitis, fatty liver disease, or dyslipidemia. Examples of dyslipidemia may include subjects having elevated cholesterol levels, elevated triglyceride levels, and/or reduced HDL/LDL ratios.

“Overweight” and “Obesity” mean an abnormal or excessive fat accumulation that presents a risk to health. “Overweight” refers to a subject with a weight that is higher than what is considered a healthy weight for a given height and a body mass index above a first threshold and below a second threshold. The second threshold may be understood to be larger than the first threshold. For a human subject, the first threshold of a body mass index may be 25.0 and the second threshold of a body mass index may be 30. “Obesity” refers to a subject with a weight that is also higher than what is considered a health weight for a given height and a body mass index above a second threshold.

“Type 2 diabetes” means a condition where a subject is unable to process insulin to regulate blood sugar levels. “Type 2 diabetes” may be understood to develop from overweight and/or obesity or be associated with overweight and/or obesity. Insulin resistance may be measured by a known assay (e.g., Homeostatic model assessment (HOMA), Glucose/insulin ratio, Insulin sensitivity test, Insulin tolerance test, hyperinsulinemic-euglycemic clamp, or any other known assay to determine insulin resistance).

“Isomers” means compounds having the same number and kind of atoms, and hence the same molecular weight, but differing with respect to the arrangement or configuration of the atoms in space.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as mixtures of enantiomers and diastereomers, for example, as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the disclosure from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.). Chemical names were generated using PerkinElmer ChemDraw® Professional, version 17.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbol “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(+)” in nomenclature, but the skilled artisan will recognize that a structure may denote a chiral center implicitly. In some embodiments, an enantiomer or stereoisomer may be provided substantially free of the corresponding enantiomer.

The present disclosure provides, in one aspect, a pharmaceutical composition comprising a therapeutically-effective amount of a mixture of MEAI or a salt thereof and at least one N-acylethanolamine or a salt thereof.

The present disclosure provides, in another aspect, a pharmaceutical composition comprising a therapeutically-effective amount of a mixture of MEAI or a salt thereof and at least one N-acylethanolamine or a salt thereof, wherein the molar ratio between the MEAI and the N-acylethanolamine is between about 1:0.2 to about 1:2000.

As used herein, a “pharmaceutical composition” refers to a preparation of the active agents described herein with other chemical components such as physiologically suitable carriers and excipients. The purpose of a pharmaceutical composition is to facilitate administration of a compound to an organism. As used herein, the phrase “pharmaceutically acceptable carrier” refers to a carrier, an excipient or a diluent that does not cause significant irritation to an organism and does not abrogate the biological activity and properties of the administered compound. An adjuvant is included under these phrases.

The term “excipient” as used herein refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, oils such as vegetable oils or fish oils, and polyethylene glycols.

The term “carrier” as used herein refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The phrase “pharmaceutically acceptable” as used herein refers to molecular entities and compositions that are physiologically tolerable and do not typically produce an allergic or similar toxicity when administered to an individual. Preferably, and particularly where a formulation is used in humans, the term “pharmaceutically acceptable” may mean approved by a regulatory agency (for example, the U.S. Food and Drug Agency) or listed in a generally recognized pharmacopeia for use in animals (e.g., the U.S. Pharmacopeia).

The term “N-acylethanolamine” as used herein generally refers to a type of fatty acid amide, lipid-derived signaling molecules, formed when one of several types of acyl group is linked to the nitrogen atom of ethanolamine. These amides conceptually can be formed from a fatty acid and ethanolamine with the release of a molecule of water, but the known biological synthesis uses a specific phospholipase D to cleave the phospholipid unit from N-acylphosphatidylethanolamines. The suffixes-amine and -amide in these names each refer to the single nitrogen atom of ethanolamine that links the compound together: it is termed “amine” in ethanolamine because it is considered as a free terminal nitrogen in that subunit, while it is termed “amide” when it is considered in association with the adjacent carbonyl group of the acyl subunit. Names for these compounds may be encountered with either “amide” or “amine” in the present application. The term “ethanolamine” is used in the generic sense and is meant to include mono-ethanolamine, di-ethanolamine, tri-ethanolamine, and mixtures thereof.

The term “derivative” as used herein means a compound whose core structure is the same as, or closely resembles that of an N-acylethanolamine compound, but which has a chemical or physical modification, such as different or additional side groups.

The term “salt” as used herein refers to any form of an active ingredient in which the active ingredient assumes an ionic form and is coupled to a counter ion (a cation or anion) or is in solution. This also includes complexes of the active ingredient with other molecules and ions, in particular complexes which are complexed by ion interaction. Pharmaceutically acceptable salts are known to persons of ordinary skill in the art.

In certain embodiments, the molar ratio between the MEAI and the N-acylethanolamine is between about 1:0.2 to about 1:1000. In certain embodiments, the molar ratio between the MEAI and the N-acylethanolamine is between about 1:0.2 to about 1:900, about 1:0.2 to about 1:800, about 1:0.2 to about 1:700, about 1:0.2 to about 1:600, about 1:0.2 to about 1:500, about 1:0.2 to about 1:400, about 1:0.2 to about 1:300, about 1:0.2 to about 1:200, about 1:0.2 to about 1:100, about 1:0.2 to about 1:50, about 1:0.2 to about 1:40, about 1:0.2 to about 1:30, about 1:0.2 to about 1:20, or about 1:0.2 to about 1:10. Each possibility represents a separate embodiment of the present disclosure.

In certain embodiments, the molar ratio between the MEAI and the N-acylethanolamine is between about 1:0.5 to about 1:2000. In certain embodiments, the molar ratio between the MEAI and the N-acylethanolamine is between about 1:0.5 to about 1:1000, about 1:0.5 to about 1:900, about 1:0.5 to about 1:800, about 1:0.5 to about 1:700, about 1:0.5 to about 1:600, about 1:0.5 to about 1:500, about 1:0.5 to about 1:400, about 1:0.5 to about 1:300, about 1:0.5 to about 1:200, about 1:0.5 to about 1:100, about 1:0.5 to about 1:50, about 1:0.5 to about 1:40, about 1:0.5 to about 1:30, about 1:0.5 to about 1:20, or about 1:0.5 to about 1:10. Each possibility represents a separate embodiment of the present disclosure.

In certain embodiments, the molar ratio between the MEAI and the N-acylethanolamine is between about 1:1 to about 1:2000. In certain embodiments, the molar ratio between the MEAI and the N-acylethanolamine is between about 1:1 to about 1:1000, about 1:1 to about 1:900, about 1:1 to about 1:800, about 1:1 to about 1:700, about 1:1 to about 1:600, about 1:1 to about 1:500, about 1:1 to about 1:400, about 1:1 to about 1:300, about 1:1 to about 1:200, about 1:1 to about 1:100, about 1:1 to about 1:50, about 1:1 to about 1:40, about 1:1 to about 1:30, about 1:1 to about 1:20, or about 1:1 to about 1:10. Each possibility represents a separate embodiment of the present disclosure.

In certain embodiments, the pharmaceutical composition comprises about 0.5-10 mg MEAI or a salt thereof. In certain embodiments, the pharmaceutical composition comprises about 1-9.5 mg, about 1.5-9 mg, about 2-8.5 mg, about 2.5-8 mg, about 3-7.5 mg, about 3.5-7 mg, about 4-6.5 mg, about 4.5-6 mg or about 5-5.5 mg MEAI or a salt thereof. In certain embodiments, the pharmaceutical composition comprises about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg or about 10 mg MEAI or a salt thereof. Each possibility represents a separate embodiment of the present invention. In certain embodiments, the pharmaceutical composition comprises less than about 0.5 mg, less than about 1 mg, less than about 1.5 mg, less than about 2 mg, less than about 2.5 mg, less than about 3 mg, less than about 3.5 mg, less than about 4 mg, less than about 4.5 mg, less than about 5 mg, less than about 5.5 mg, less than about 6 mg, less than about 6.5 mg, less than about 7 mg, less than about 7.5 mg, less than about 8 mg, less than about 8.5 mg, less than about 9 mg, less than about 9.5 mg or about 10 mg MEAI or a salt thereof. Each possibility represents a separate embodiment of the present invention. In certain embodiments, the pharmaceutical composition comprises about 0.5 mg to about 1 mg, about 0.5 mg to about 1.5 mg, about 0.5 mg to about 2 mg, about 0.5 mg to about 2.5 mg, about 0.5 mg to about 3 mg, about 0.5 mg to about 3.5 mg, about 0.5 mg to about 4 mg, about 0.5 mg to about 4.5 mg, about 0.5 mg to about 5 mg, about 0.5 mg to about 5.5 mg, about 0.5 mg to about 6 mg, about 0.5 mg to about 6.5 mg, about 0.5 mg to about 7 mg, about 0.5 mg to about 7.5 mg, about 0.5 mg to about 8 mg, about 0.5 mg to about 8.5 mg, about 0.5 mg to about 9 mg or about 0.5 mg to about 9.5 mg MEAI or a salt thereof. Each possibility represents a separate embodiment of the present disclosure.

In certain embodiments, the pharmaceutical composition comprises about 200-1800 mg N-acylethanolamine or a salt thereof. In certain embodiments, the pharmaceutical composition comprises about 250-1550 mg, about 300-1200 mg, about 350-950 mg, about 400-700 mg, about 450-600 mg or about 500-550 mg N-acylethanolamine or a salt thereof. Each possibility represents a separate embodiment of the present disclosure. In certain embodiments, the pharmaceutical composition comprises at least about 50 mg, at least about 100 mg, at least about 150 mg, at least about 200 mg, at least about 250 mg, at least about 300 mg, at least about 350 mg, at least about 400, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, at least about 1000 mg, at least about 1050 mg, at least about 1100 mg, at least about 1150 mg, at least about 1200 mg, at least about 1250 mg, at least about 1300 mg, at least about 1350 mg, at least about 1400 mg, at least about 1450 mg, at least about 1500 mg, at least about 1550 mg, at least about 1600 mg, at least about 1650 mg, at least about 1700 mg, at least about 1750 mg or at least about 1800 mg N-acylethanolamine or a salt thereof. In certain embodiments, the pharmaceutical composition comprises about 50 mg, about 100 mg, about 150 mg, about 200 mg, about 250 mg, about 300 mg, about 350 mg, about 400, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg or about 1800 mg N-acylethanolamine or a salt thereof. Each possibility represents a separate embodiment of the present disclosure.

In some embodiments, the pharmaceutical composition comprising MEAI also comprises a concentration of N-acylethanolamine or pharmaceutically acceptable salt thereof sufficient to provide a patient with a dose of the N-acylethanolamine or a salt thereof of about 2.5 mg/kg/day to about 36 mg/kg/day body weight. In certain embodiments, the pharmaceutical composition comprises a concentration of N-acylethanolamine or pharmaceutically acceptable salt thereof sufficient to provide a patient with a dose of about 2.5 to about 5 mg/kg/day, about 5 mg/kg/day to about 7.5 mg/kg/day, about 7.5 mg/kg/day to about 10 mg/kg/day, about 10 mg/kg/day to about 12.5 mg/kg/day, about 12.5 mg/kg/day to about 15 mg/kg/day, about 15 mg/kg/day to about 17.5 mg/kg/day, about 17.5 mg/kg/day to about 20 mg/kg/day, about 20 mg/kg/day to about 22.5 mg/kg/day, about 22.5 mg/kg/day to about 25 mg/kg/day, about 25 mg/kg/day to about 27.5 mg/kg/day, about 27.5 mg/kg/day to about 30 mg/kg/day, about 30 mg/kg/day to about 32.5 mg/kg/day, about 32.5 mg/kg/day to about 36 mg/kg body weight/day. In certain embodiments, the pharmaceutical composition comprises a concentration of N-acylethanolamine or pharmaceutically acceptable salt thereof sufficient to provide a patient with a dose of N-acylethanolamine or a salt thereof of about 2.5 mg/kg/day, about 5 mg/kg/day, about 7.5 mg/kg/day, about 10 mg/kg/day, about 12.5 mg/kg/day, about 15 mg/kg/day, about 17.5 mg/kg/day, about 20 mg/kg/day, about 22.5 mg/kg/day, about 25 mg/kg/day, about 27.5 mg/kg/day, about 30 mg/kg/day, about 32.5 mg/kg/day, or about 36 mg/kg bodyweight/day. Each possibility represents a separate embodiment of the present disclosure. In certain embodiments, the pharmaceutical composition comprises a concentration of N-acylethanolamine or pharmaceutically acceptable salt thereof sufficient to provide a patient with a dose of N-acylethanolamine or a salt thereof of about 2.5 mg/kg/day, less than about 2.5 mg/kg/day, less than about 5 mg/kg/day, less than about 7.5 mg/kg/day, less than about 10 mg/kg/day, less than about 12.5 mg/kg/day, less than about 15 mg/kg/day, less than about 17.5 mg/kg/day, less than about 20 mg/kg/day, less than about 22.5 mg/kg/day, less than about 25 mg/kg/day, less than about 27.5 mg/kg/day, less than about 30 mg/kg/day, less than about 32.5 mg/kg/day, or about 36 mg/kg bodyweight/day. Each possibility represents a separate embodiment of the present disclosure. In certain embodiments, the pharmaceutical composition comprises a concentration of N-acylethanolamine or pharmaceutically acceptable salt thereof sufficient to provide a patient with a dose of N-acylethanolamine or a salt thereof of about 2.5 mg/kg/day to about 5 mg/kg/day, about 2.5 mg/kg/day to about 7.5 mg/kg/day, about 2.5 mg/kg to about 10 mg/kg/day, about 2.5 mg/kg/day to about 12.5 mg/kg/day, about 2.5 mg/kg/day to about 15 mg/kg/day, about 2.5 mg/kg/day to about 17.5 mg/kg/day, about 2.5 mg/kg/day to about 20 mg/kg/day, about 2.5 mg/kg/day to about 22.5 mg/kg/day, about 2.5 mg/kg/day to about 25 mg/kg/day, about 2.5 mg/kg/day to about 27.5 mg/kg/day, about 2.5 mg/kg/day to about 30 mg/kg/day, about 2.5 mg/kg/day to about 32.5 mg/kg/day, or about 2.5 mg/kg/day to about 36 mg/kg body weight/day. Each possibility represents a separate embodiment of the present disclosure.

In certain embodiments, the N-acylethanolamine is N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), or salts thereof, or any combination thereof. Each possibility represents a separate embodiment of the present invention. In certain embodiments, the N-acylethanolamine is PEA or a salt thereof. In certain embodiments, the N-acylethanolamine consists of PEA or a salt thereof. In certain embodiments, the N-acylethanolamine consists of PEA.

In some embodiments, administering a pharmaceutical composition comprising an N-acylethanolamine or a pharmaceutically acceptable salt thereof and separately, concurrently, or simultaneously administering a pharmaceutical composition comprising 5-methoxy-2-aminoindan may improve the therapeutic potency of the separate, concurrent, or simultaneous administration compared to administering a pharmaceutical composition comprising 5-methoxy-2-aminoindan alone. In some embodiments, a required therapeutic dosage of 5-methoxy-2-aminoindan may be decreased when administered with an N-acylethanolamine compared to administering 5-methoxy-2-aminoindan alone.

In certain embodiments, the pharmaceutical composition is formulated for systemic administration. In certain embodiments, the pharmaceutical composition is formulated for oral, oral mucosal, nasal, sublingual, inhalational, topical, rectal, vaginal, parenteral, intravenous, intramuscular, or subcutaneous administration. In certain embodiments, the pharmaceutical composition is formulated for oral, oral mucosal, nasal, or sublingual administration. Each possibility represents a separate embodiment of the present invention. In certain embodiments, the pharmaceutical composition is formulated for oral administration. In certain embodiments, the pharmaceutical composition is formulated for oral mucosal administration. In certain embodiments, the pharmaceutical composition is formulated for nasal administration. In certain embodiments, the pharmaceutical composition is formulated for sublingual administration.

Techniques for formulation and administration of drugs are well known in the art, and may be found, e.g. in “Remington's Pharmaceutical Sciences,” Mack Publishing Co., Easton, Pa. Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

The term “oral administration” refers to any method of administration in which an active agent can be administered by swallowing, chewing, sucking, or drinking an oral dosage form. Examples of solid dosage forms include conventional tablets, multi-layer tablets, capsules, caplets, etc., which do not substantially release the drug in the mouth or in the oral cavity.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include stiff or soft, sealed capsules made of gelatin and a plasticizer, such as glycerol or sorbitol. The capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration. For buccal and sublingual administration, the compositions may take the form of tablets or lozenges formulated in conventional manner or in adhesive carriers. Alternatively, the active ingredient may be in powder form for constitution with a suitable vehicle, e.g., a sterile, pyrogen-free, water-based solution, before use.

Pharmaceutical compositions suitable for use in the context of the present invention include compositions wherein the active ingredients are contained in an amount effective to achieve the intended purpose. More specifically, a “therapeutically effective amount” means an amount of active ingredients effective to prevent, alleviate, or ameliorate symptoms or side effects of a disease or disorder, or prolong the survival of the subject being treated. Determination of a therapeutically effective amount is well within the capability of those skilled in the art, especially in light of the detailed disclosure provided herein. More specifically, a “therapeutically effective amount of a mixture” means an amount of at least two active ingredients, wherein each one of the active ingredients independently may not be in a therapeutically effective amount or wherein both of the active ingredients may not be in a therapeutically effective amount, the mixture is nevertheless effective to prevent, alleviate, or ameliorate symptoms or side effects of a disease or disorder, or prolong the survival of the subject being treated. The term “mixture” as used herein refers to a non-covalent combination of two molecules.

For any preparation used in the methods of the invention, the dosage or the therapeutically effective amount can be estimated initially from in vitro, in vivo and cell culture assays. For example, a dose can be formulated in animal models to achieve a desired concentration or titer. Such information can be used to more accurately determine useful doses in humans. The dosage of each compound of the claimed combinations depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used. Continuous daily dosing may not be required; a therapeutic regimen may require cycles, during which time a drug is not administered, or therapy may be provided on an as-needed basis during periods of acute disease worsening. Dosage escalation may or may not be required; a therapeutic regimen may require reduction in medication dosage. Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition (See, e.g., Fingl, E. et al. (1975), “The Pharmacological Basis of Therapeutics,” Ch. 1, p. 1). Depending on the severity and responsiveness of the condition to be treated, dosing can be of a single or a plurality of administrations, with course of treatment lasting from several days to several weeks, or until cure is affected or until a desired level of diminution of the disease state is achieved.

The present disclosure further provides, in another aspect, a dosage unit comprising or consisting of the pharmaceutical composition described above.

In certain embodiments, the dosage unit comprises the pharmaceutical composition described above. In certain embodiments, the dosage unit consisting of the pharmaceutical composition described above. In certain embodiments, the dosage unit is formulated as a gel, a powder, or a spray. In certain embodiments, the dosage unit is formulated as a gel. In certain embodiments, the dosage unit is formulated as a powder.

In certain embodiments, the dosage unit is formulated as a spray.

The present disclosure further provides, in another aspect, a pharmaceutical composition or a dosage unit as described above for use in a method for preventing or treating a condition amenable to prevention or treatment by at least one MEAI.

The term “treating” as used herein, includes, but is not limited to, any one or more of the following: abrogating, ameliorating, inhibiting, attenuating, reducing, blocking, suppressing, reducing, delaying, halting, alleviating, preventing, or slowing the onset of one or more symptoms or side effects of the diseases or conditions of the invention.

The term “acute” refers to a condition or treatment with a relatively short time course.

The term “chronic” as used herein means that the length of time of the diseases or conditions or treatment of the invention can be weeks, months, or possibly years. The intensity of the diseases or conditions can differentiate according to various conditions such as patient age, temperature, season, type of disease, etc.

The term “about” as used herein in relation to a value, a plurality of values or a range of values defined by a lowest and highest values means a value which is 10% lower and/or higher than the corresponding value, plurality of values or range of values. For example, the phrase “about 1” means “0.9 to 1.1”, the phrase “about 1 or 2” means “0.9 to 1.1 or 1.8 to 2.2”, and the phrase “about 1 to about 2” means “0.9 to 2.2”.

As used herein, the singular form “a”, “an” and “the” include plural references unless the context clearly dictates otherwise. For example, the term “a compound” or “at least one compound” may include a plurality of compounds, including mixtures thereof.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50 (4): 219-244 (1966) and the following Table for Equivalent Surface Area Dosage Factors).

TABLE 2
Equivalent Surface Area Dosage Factors.

To:

| | | | | | Adult |
|---|---|---|---|---|---|
| | Mouse | Rat | Monkey | Dog | Human |
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure is not limited to the particular embodiment disclosed, but that the present disclosure will include all embodiments falling within the scope of the appended claims.

The following example is presented to more fully illustrate some embodiments of the disclosure. It should in no way be construed, however, as limiting the broad scope of the disclosure.

### Example 1:5-Methoxy-2-Aminoindane (MEAI) Anti-Obesity Effect

### Methods

Mice. The experimental protocol used was approved by the Institutional Animal Care and Use Committee of the Hebrew University, which is an AAALAC International accredited institute. Male 6-week-old C57BL/6 mice were obtained from Envigo. Animal studies were conducted in compliance with the ARRIVE guidelines, which aim to improve the transparency and reproducibility of preclinical research. The principle of replacement, refinement, or reduction was followed to minimize the number of animals used in this study. All the animals were housed in specific pathogen-free (SPF) conditions, with no more than five animals of the same gender and dose group per cage, in standard plastic cages, with natural soft sawdust provided as bedding.

Male 6-week-old C57BL/6 mice (Envigo, Israel) were maintained under a 12-h light/dark cycle and fed ad libitum. A total of 32 animals were divided into four experimental groups (N=8 mice per group) receiving single doses of 40, 60, or 100 mg/kg of MEAI or vehicle (sterile water), which was administered via oral gavage two hours prior to the dark phase. The animals were monitored for 48 hours post-dose for drug tolerability, food and water intakes as well as activity and metabolic parameters. At the end of the experiment, the animals were euthanized, and tissues (brain, liver, fat, and kidney) and blood were collected and stored frozen for future analyses. To generate diet-induced obesity, C57BI6/J mice were fed either a high-fat diet (HFD) (60% of calories from fat, 20% from protein, and 20% from carbohydrates; Research Diet, D12492) or a standard laboratory diet (STD, 14% fat, 24% protein, 62% carbohydrates; NIH-31 rodent diet) for 18 weeks.

Effect of MEAI on Obesity. Male C57BL/6 mice were used to establish DIO by feeding them a high-fat diet (HFD; 60% Kcal fat, 20% Kcal protein, and 20% Kcal carbohydrates; Research Diet, D12492) for 18 weeks. After this period, mice were treated with either vehicle (sterile water, N=8) or MEAI (N=11) daily for 28 days by gavage at a dose of 40 mg/kg/day. Age-matched control mice (N=10) on a standard diet (STD; 14% Kcal fat, 24% Kcal protein, 62% Kcal carbohydrates; NIH-31 rodent diet) and received vehicle daily. The body weight of all mice was monitored daily, and total body fat and lean mass were determined by EchoMRI-100H™ (Echo Medical Systems LLC, Houston, TX, USA). On Day 29, at the end of the experimental period, mice were euthanized by a cervical dislocation under anesthesia. The kidneys, brain, liver, and fat pads were removed and weighed, and samples were either snap-frozen or fixed in buffered 4% formalin. Trunk blood was collected to determine biochemical parameters.

Sucrose Preference Test. Thirteen-week-old male C57BL/6 mice maintained on a STD and housed individually were habituated to two water bottles in their home cage for 48 h prior to the test. Baseline intake was measured by weighing the bottles. On the test day (Day 1), 2 h before the onset of the dark phase, fresh water and a 1.5% sucrose solution were added to the bottles, and mice were subsequently treated orally with MEAI (40 mg/kg, N=8) or sterile water (N=8). The mice were allowed to drink from either bottle freely for 24 h, after which the bottles were weighed to measure consumption. The study was repeated for an additional day (Day 2), with the bottles switched in position (in the cage) to account for side preference. The sucrose and water intake over the two days were averaged, and the sucrose preference index was calculated as the average consumed sucrose solution divided by the average volume of total consumed liquid (average water plus average sucrose solution).

Multi-parameter metabolic assessment. The metabolic profiles and food and water intakes of the mice were assessed by using the Promethion High-Definition Behavioral Phenotyping System (Sable Instruments, Inc., Las Vegas, NV, USA). Data acquisition and instrument control were performed using MetaScreen software version 2.2.18.0, and the obtained raw data were processed using ExpeData version 1.8.4 using an analysis script detailing all aspects of data transformation. Mice with free access to food and water were subjected to a standard 12 h light/12 h dark cycle, which consisted of a 48 h acclimation period followed by 24 h of sampling. Respiratory gases were measured by using the GA-3 gas analyzer (Sable Systems, Inc., Las Vegas, NV, USA) using a pull-mode, negative pressure system. Air flow was measured and controlled by FR-8 (Sable Systems, Inc., Las Vegas, NV, USA), with a set flow rate of 2000 mL/min. Water vapor was continuously measured and its dilution effect on O2 and CO2 was mathematically compensated. Respiratory exchange rate (RER) was calculated as the ratio of CO₂ produced (VCO₂) to O₂ consumed (VO₂) using Equation (1):

$$RER = {VCO}_{2} / {VO}_{2} \qquad ( 1 )$$

Total energy expenditure (TEE) was calculated using VO₂ and RER, according to Equation (2):

$$TEE = {VO}_{2} \times ( 3.815 + 1.232 \times RER ) \qquad ( 2 )$$

Fat oxidation (FO) and carbohydrate oxidation (CHO) were calculated using VO₂ and VCO₂ based on Equations (3) and (4), respectively:

$$FO = 1.69 \times {VO}_{2} - 1.69 \times {VCO}_{2} \qquad ( 3 )$$

$$CHO = 4.57 \times {VCO}_{2} - 3.23 \times {VO}_{2} \qquad ( 4 )$$

Wheel running and locomotive activity. The assessment of wheel running and locomotor activity was performed using the Promethion High-Definition Behavioral Phenotyping System (Sable Instruments, Inc., Las Vegas, NV, USA). Wheel revolutions were measured with a monitor that recorded voluntary wheel running activity, and locomotor activity was quantified using disruptions of infrared XYZ beam arrays, with a beam spacing of 0.25 cm.

Glucose Tolerance Test (ipGTT) and the Insulin Tolerance Test (ipITT). On day 25 of the experiment, mice were subjected to an overnight fasting and then injected with glucose (1.5 g/kg i.p.) on the following day (day 26). Blood glucose levels were determined at 0, 15, 30, 45, 60, 90, and 120 min after injection using the Contour® glucometer (Bayer, Pittsburgh, PA, USA). The mice were then fasted for 6 h on the next day (day 27) before being administered insulin (0.75 U/kg, i.p.; Actrapid vials, Novo Nordisk A/S, Bagsværd, Denmark). Blood glucose levels were determined at the same intervals as described above. To assess insulin resistance, the homeostasis model assessment insulin resistance (HOMA-IR) was calculated as fasting serum insulin ([μU/mL]×fasting plasma glucose [mmol/L]/22.5). The relative insulin sensitivity index (ISI) was calculated as 1/(glucose×insulin)×1000, with glucose expressed as mg/dl and insulin as mU/L.

Blood and urine biochemistry. Serum levels of alanine aminotransferase (ALT), aspartate aminotransferase (AST), alkaline phosphatase (ALP), cholesterol, triglycerides, high-density lipoprotein (HDL), and low-density lipoprotein (LDL) were determined by using the Cobas C-111 chemistry analyzer (Roche, Switzerland). Blood urea nitrogen (BUN) was calculated based on serum urea levels as follows: BUN (mg/dL)=Urea (mg/dL)/2.1428. Fasting blood glucose was measured using the Contour® glucometer (Bayer, Pittsburgh, PA). Serum insulin was determined using an Ultra-Sensitive Mouse Insulin ELISA kit (Crystal Chem, Inc., Elk Grove Village, IL, USA).

Hepatic Triglyceride and Cholesterol Content. Liver tissue was extracted as described in (Tam et al., 2012), and its cholesterol and triglycerides contents were determined using a Cobas C-111 chemistry analyzer (Roche, Switzerland).

Histopathology. First, 5 μm paraffin-embedded liver sections from 5 animals per group were stained with hematoxylin-eosin staining. Liver images were captured using a Zeiss Axio Scope A1 light microscope (Carl Zeiss AG, Jena, Germany equipped with a Zeiss AxioCam ICc5 color camera. Ten random 40× fields of view were taken from each animal to obtained representative images.

Oil Red O staining. Liver cryosections (8 μm) were stained with Oil Red O (Cat #ab150678; Abcam) following the manufacturer's protocol. Images were acquired as described above. For quantitative analysis of Oil Red O staining, the area of lipid droplets in the liver cryosections was measured using the ImageJ software.

Statistics. The data are presented as mean±SEM. Statistical analysis was conducted using GraphPad Prism 6.0 software (GraphPad Software, CA, USA). Differences between the two groups were determined using an unpaired two-tailed Student's t-test. For comparisons involving multiple groups and time-dependent variables, ANOVA was employed, followed by Tukey's multiple comparisons test. Statistical significance was considered when p-values were less than 0.05.

### Results

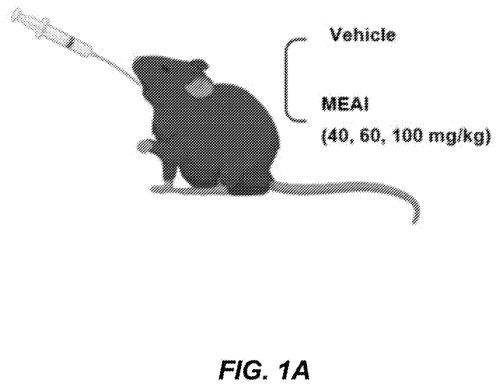

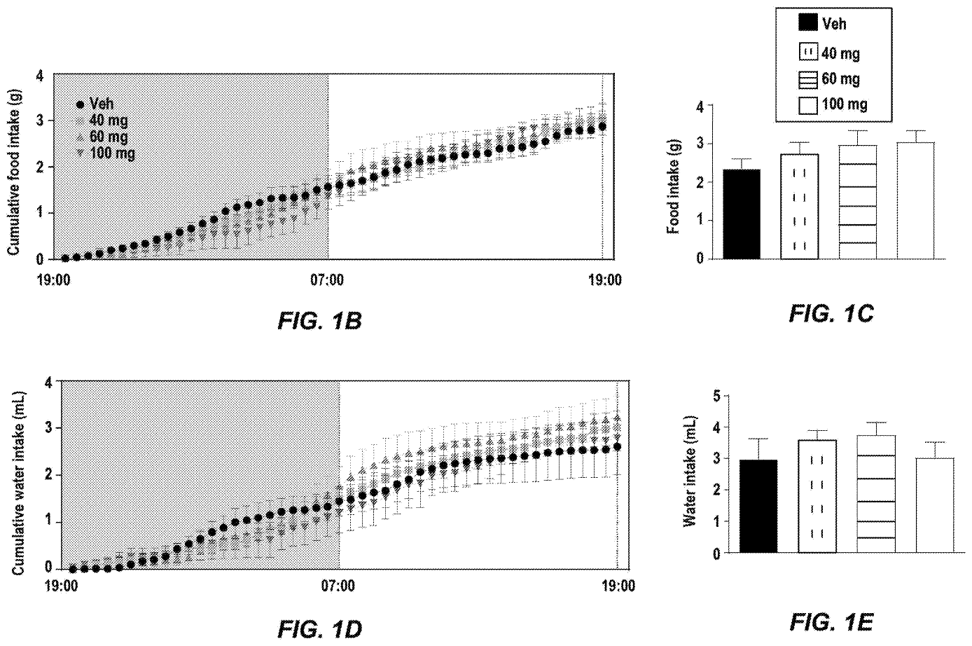

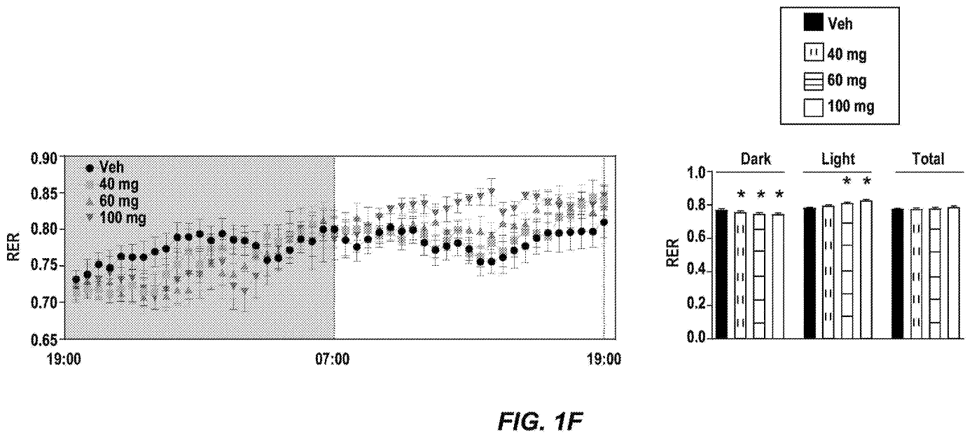

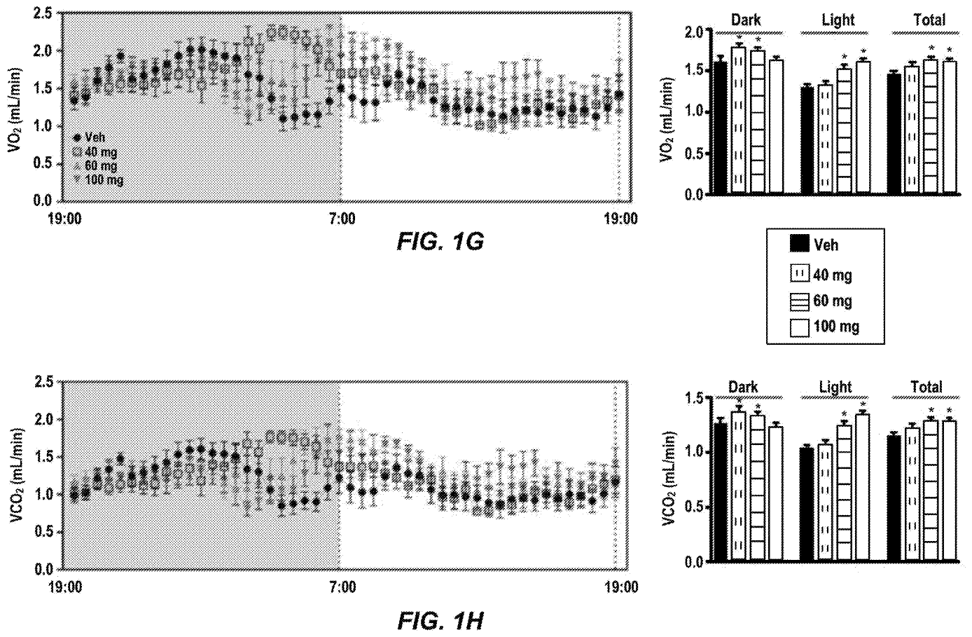

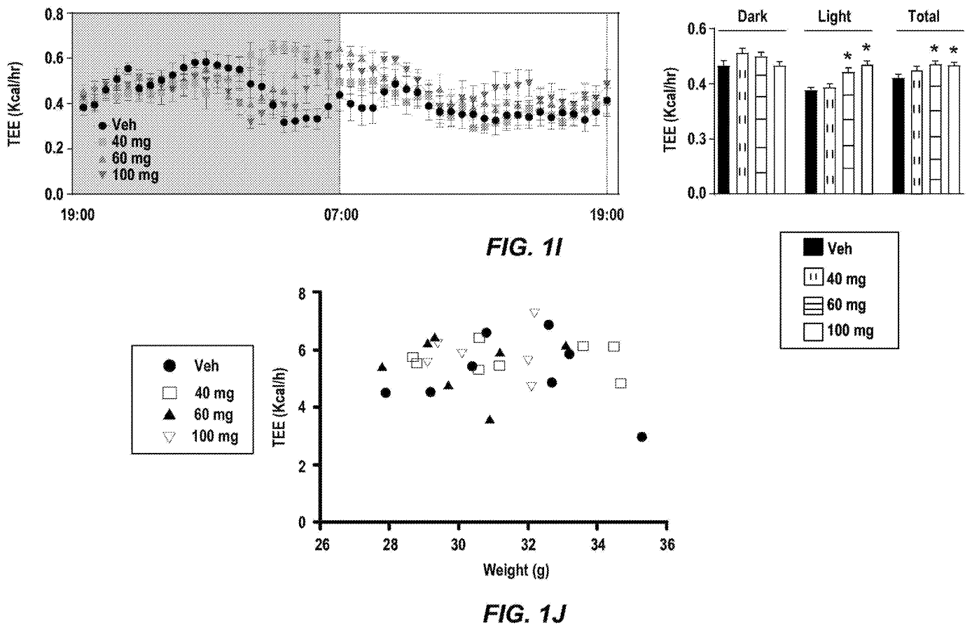

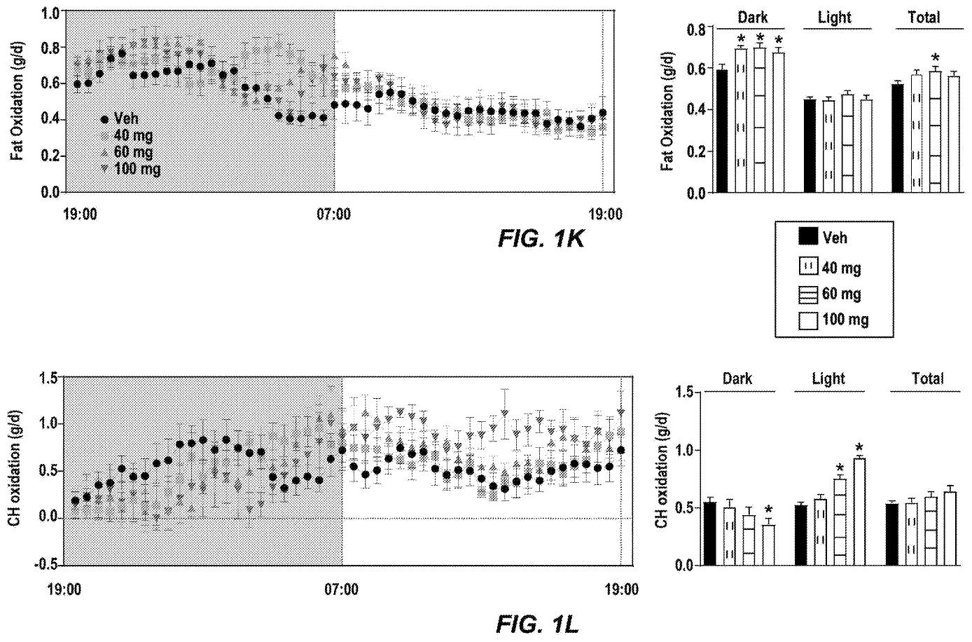

To assess the immediate effects of MEAI on food intake patterns and respirometric parameters, a single dose of 40, 60, or 100 mg/kg was administered two hours prior to the onset of the dark phase, as depicted in FIG. 1A. The results indicated that the drug was well-tolerated, with no observable changes in behavior at the 40 and 60 mg/kg dosages. However, two subjects in the 100 mg/kg group died within a few hours of the drug administration, suggesting reduced tolerance at this dose in combination with the stress caused by metabolic testing, and thus they were excluded from the analysis. Minor changes in feeding patterns were observed during the active (dark) and inactive (light) phases following MEAI administration, but these changes did not reach statistical significance (FIG. 1B, C). Moreover, there were no notable changes in water consumption (FIG. 1D, E). In contrast, acute MEAI administration resulted in significant changes in respirometric parameters. The increase in RER during the light phase at the 60 and 100 mg doses (FIG. 1F) was attributed to higher rates of oxygen consumption (VO₂) and carbon dioxide emission (VCO₂) (as demonstrated in FIG. 1G). Across the 24-hour period examined, significant dose-dependent increases in TEE (FIG. 1I, J) and corresponding elevations in FO and CHO rates (FIG. 1K, L) were evident, indicating changes in the energy profile.

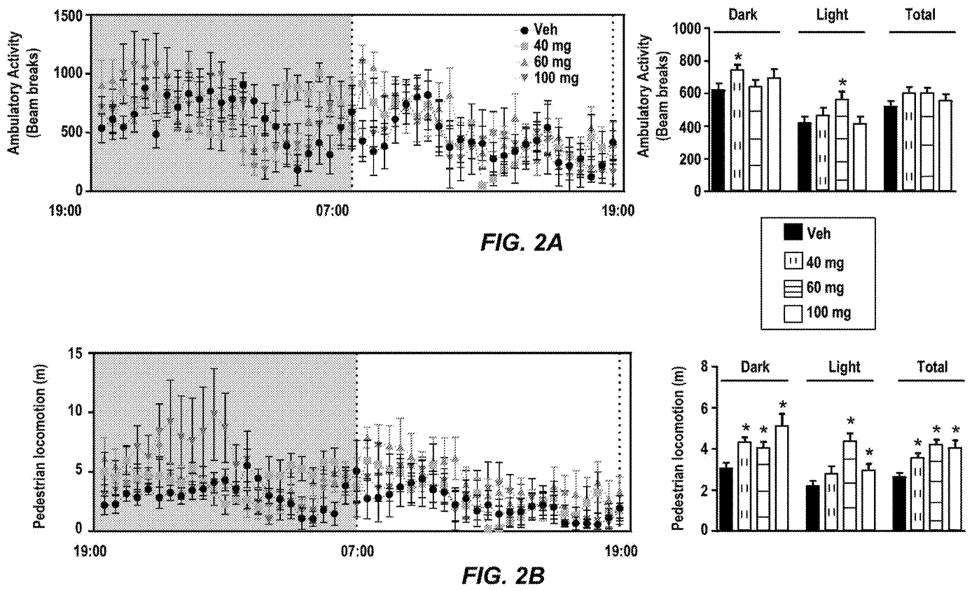

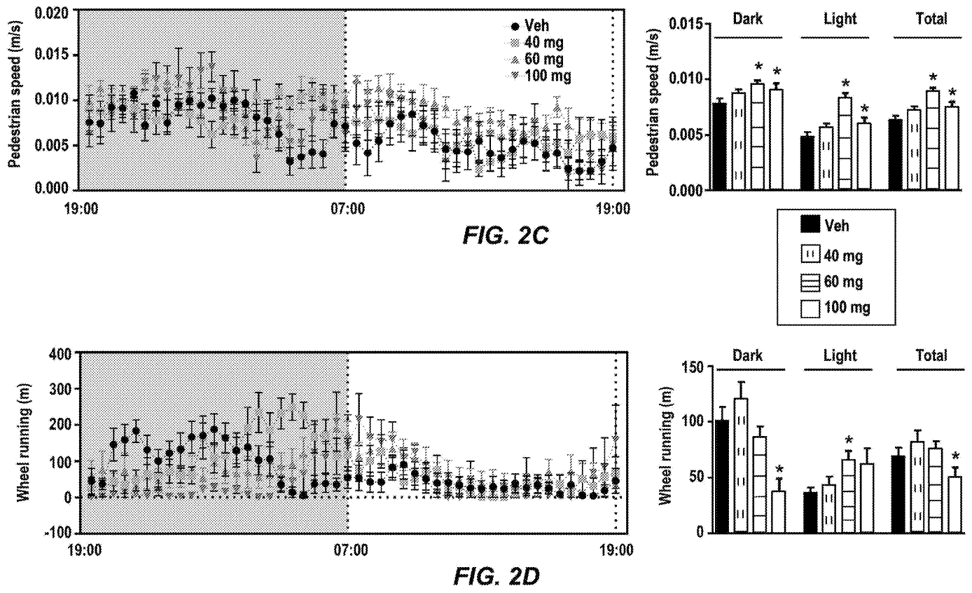

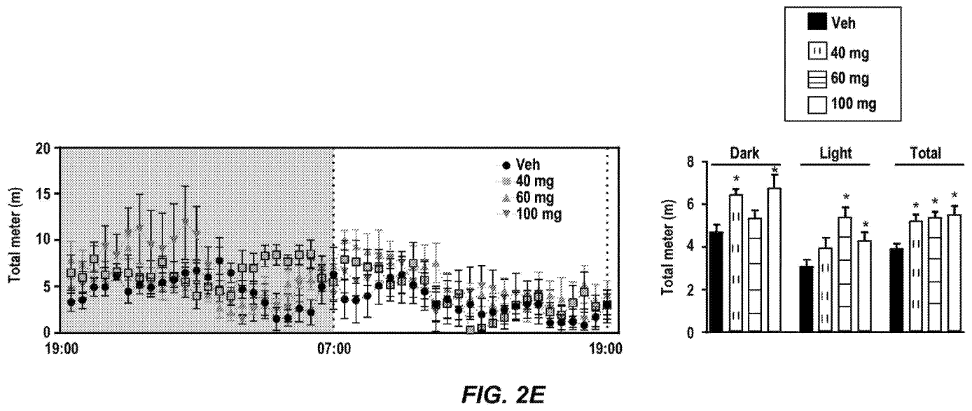

Next, the acute impact of MEAI on patterns on activity was investigated. Overall, there were no significant changes in the total number of beam breaks recorded, which represents the combination of ambulatory and fine movements (FIG. 2A). Notably, MEAI produced a significant and dose-dependent increase in directed voluntary activity and speed, such as moving around the cage for feeding, drinking, and grooming, at doses of 40 mg/kg and above (FIG. 2B, C). Although there were minor elevations in voluntary wheel running pattern at 40 and 60 mg/kg, the 100 mg/kg dose group experienced a significant suppression of wheel running (FIG. 2D). Lastly, MEAI caused an increase in the total pedometer count at all doses tested (FIG. 2E).

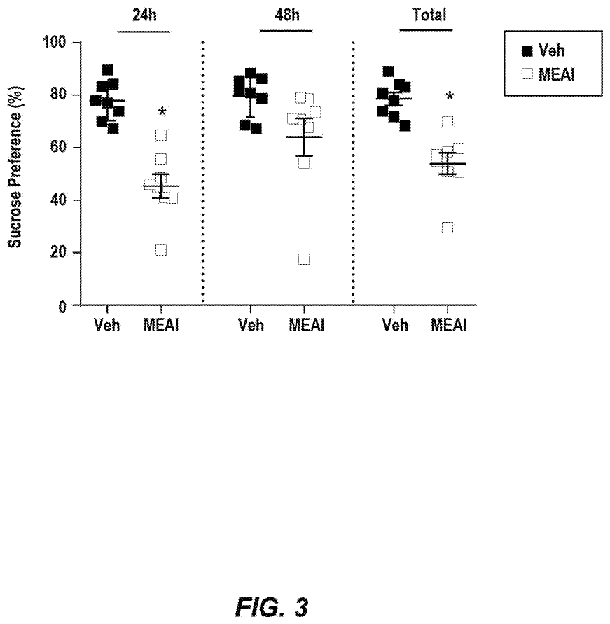

To assess the impact of MEAI on sweet taste preference, a sucrose preference test (SPT), a commonly used reward-based test to detect anhedonia, was employed. Following a single injection, MEAI at a dosage of 40 mg/kg significantly reduced the acute preference of mice for sucrose solution, without any accompanying decrease in water intake levels. This effect was most evident during the initial 24-hour period, with a slight reduction noted during the subsequent 24-hour period (FIG. 3). These results indicate that MEAI has the potential to impede reward-stimuli, leading to decreased hedonic effects that are typically associated with palatable food.

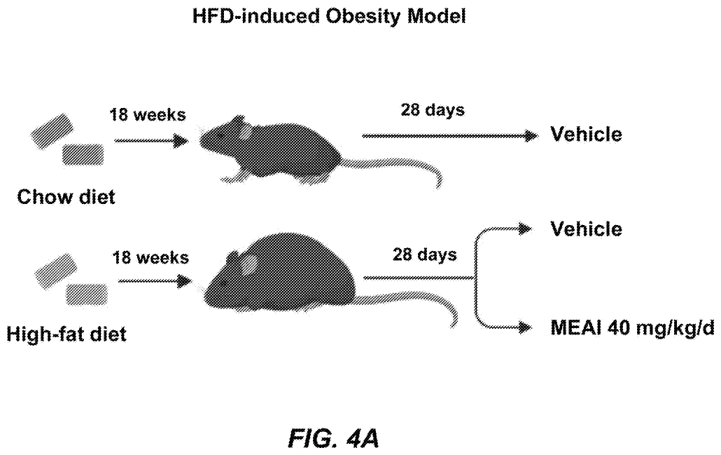

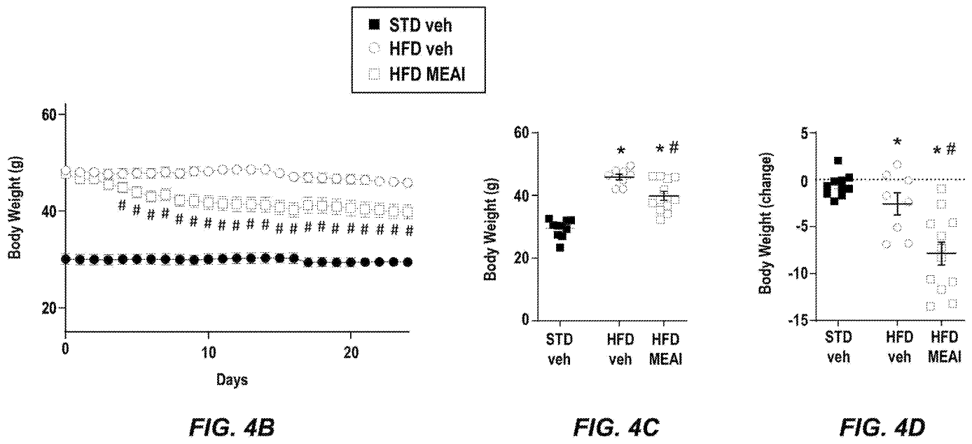

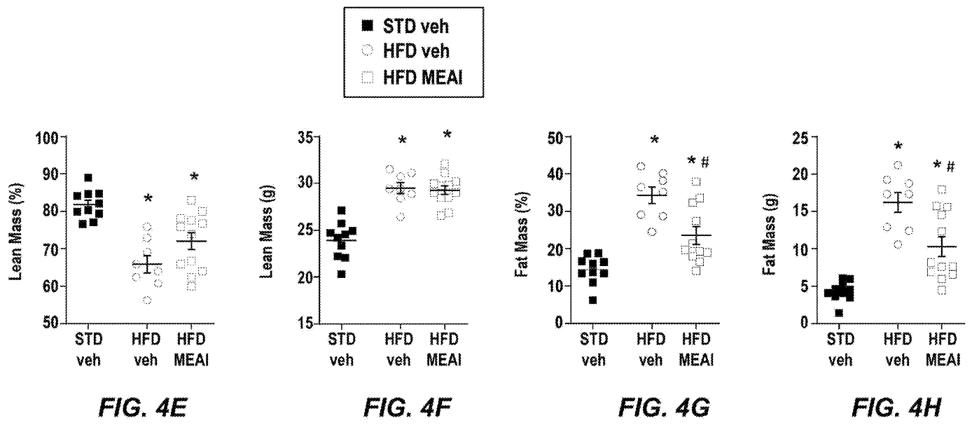

The impact of MEAI on food addiction behavior and the metabolic efficacy of MEAI in regulating appetite, treating obesity, and related abnormalities was assessed in a DIO mouse model (FIG. 4A). To evaluate the effects of a chronic exposure, a sub-optimal dose of 40 mg/kg/day of MEAI was tested in this model. At baseline prior to drug treatment, the mice fed with a HFD exhibited significantly greater weight compared to the control group fed with STD. Over the 28 days treatment period, MEAI treatment significantly reduced the body weight of HFD-fed mice (FIG. 4B), resulting in an approximate 15% decrease in total body weight compared to the obese vehicle-treated group (FIG. 4C, D). Therefore, the overweight of mice on HFD was significantly reduced by MEAI. Additionally, MEAI treatment reduced adiposity associated with obesity in the DIO model, preserved the lean body mass, the lean body mass ratio, and net lean mass (FIG. 4E, F), while simultaneously reducing the overall fat mass (FIG. 4G, H).

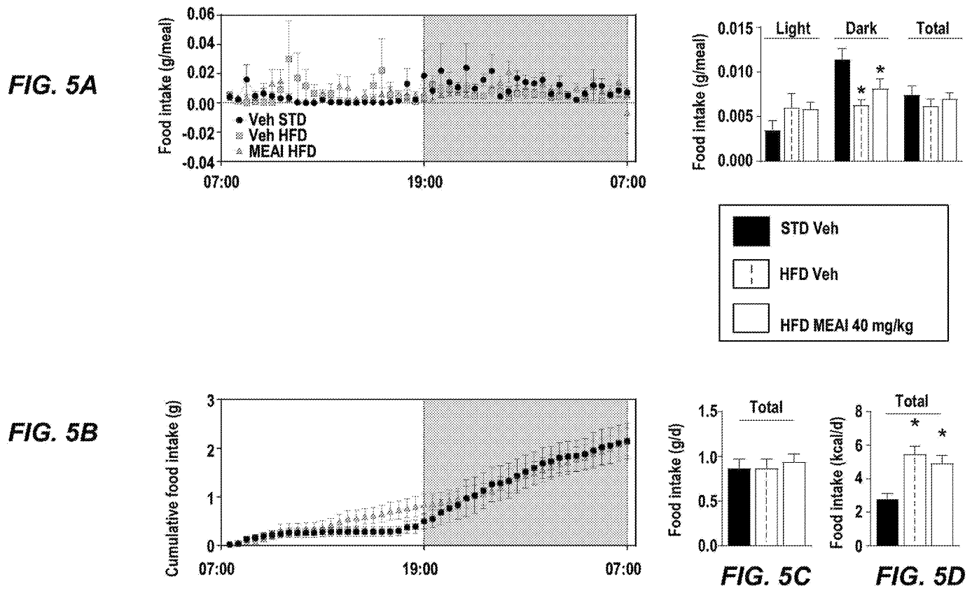

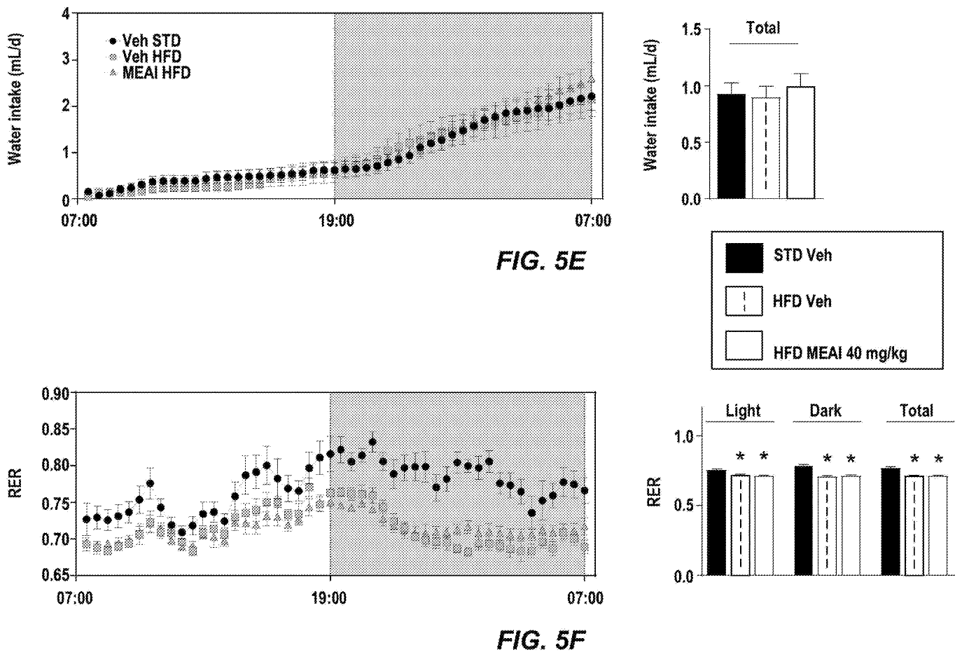

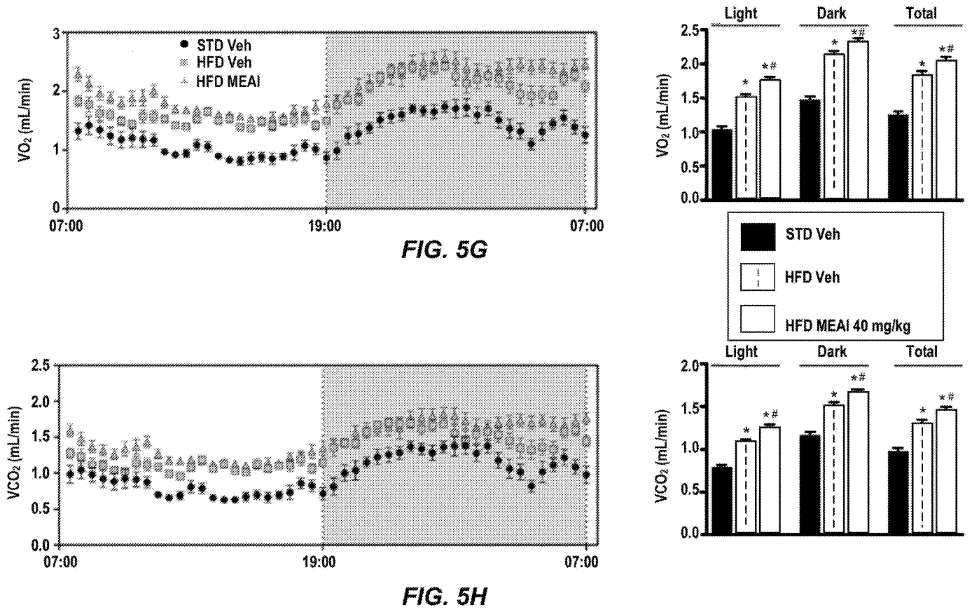

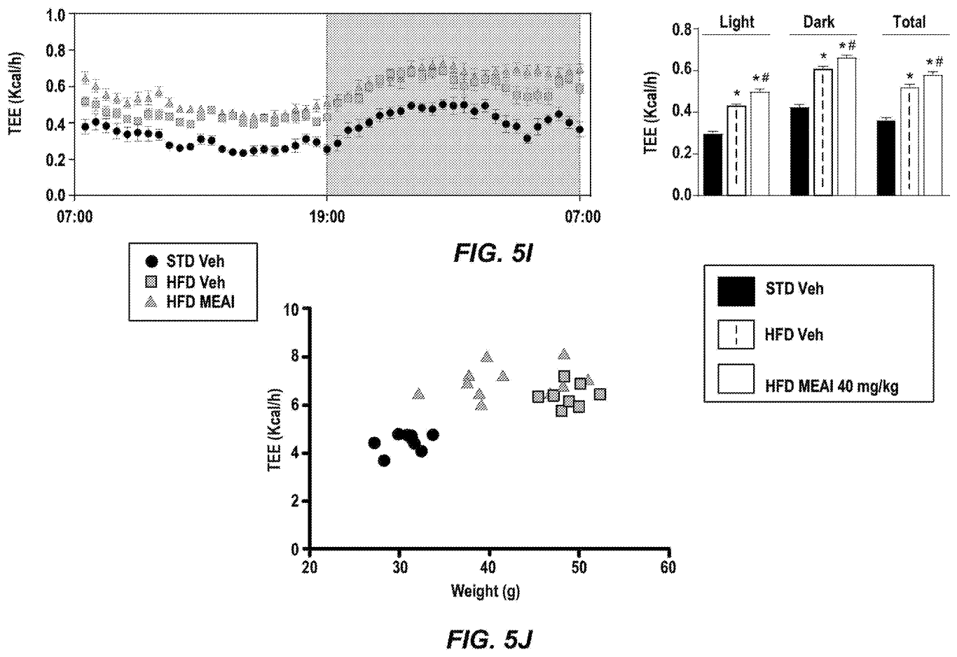

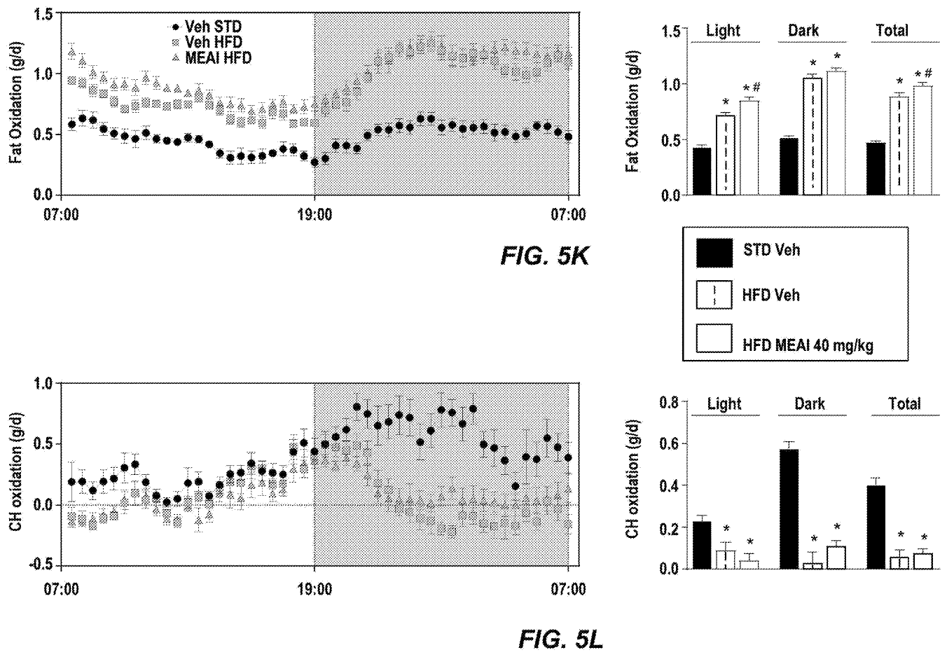

Analysis of feeding behavior revealed that mice on the HFD, both in the MEAI and vehicle treated groups, consumed smaller meals as indicated by a reduction in food intake per meal (FIG. 5A). However, the cumulative food intake over the 24-hour period was similar among all groups (FIG. 5B, C), primarily due to the higher caloric density of the HFD (FIG. 5D). Furthermore, as found in the acute settings in lean animals (FIG. 1D, E), MEAI treatment did not have any effect on water intake in the obese mice (FIG. 5E).

Metabollicaly, the RER was slightly decreased in both the HFD vehicle and MEAI-treated groups in comparison to the STD vehicle group (FIG. 5F). MEAI administration slightly increased the oxygen consumption and carbon dioxide production compared to the HFD vehicle-treated group (FIG. 5G, H). Notably, the MEAI-treated group showed a significant increase in energy expenditure compared to both the HFD and STD vehicle-treated groups, with a clear elevation observed during both light and dark phases (FIG. 5I). Regression analysis of TEE versus body mass exhibited significant differences between the groups that were independent of body mass (FIG. 5J). In addition, MEAI treatment led to an increase in the overall rate of FO compared to both the HFD- and STD-vehicle-treated groups (FIG. 5K). However, CHO was markedly reduced in both the vehicle and MEAI-treated groups, with no effect of the drug itself (FIG. 5L).

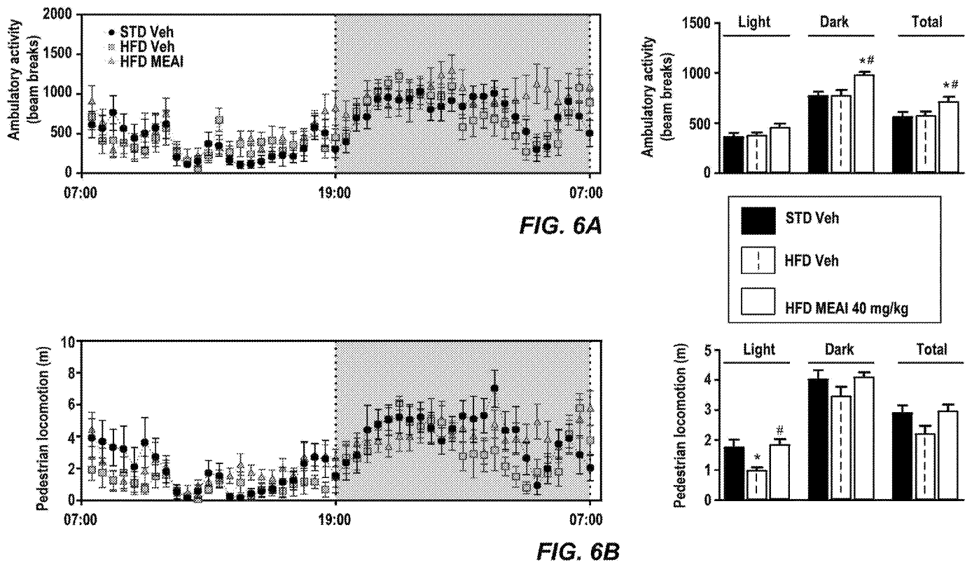

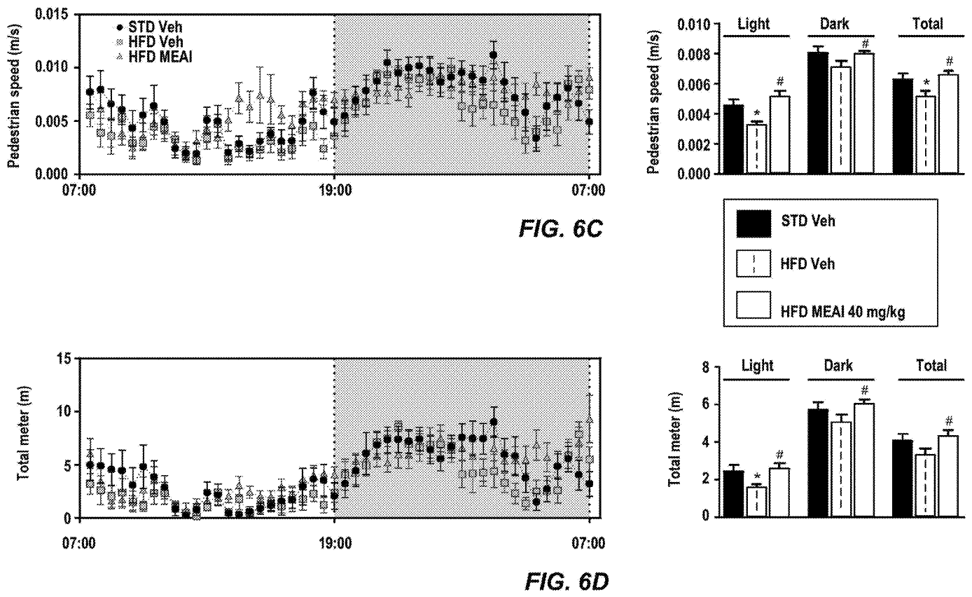

Analysis of locomotive activity indicated that MEAI treatment significantly increased voluntary actions such as pedestrian activity and grooming, throughout the day but were more prominent during the dark phase of the day, consistent with their nocturnal nature (FIG. 6A). Interestingly, while pedestrian locomotive activity, speed, and total distance travelled in the MEAI group was increased in comparison with the HFD-vehicle treated group, the drug did not surpass the levels of the STD-vehicle treated group, suggesting that it does not induce an over-stimulatory effect (FIG. 6B-D). A similar behavior pattern was observed in the wheel running parameter, which was a purely voluntary activity. MEAI-treated animals displayed an increased capability to run on the voluntary wheel, and their speed was similar to that of the STD-vehicle treated group (FIG. 6E, F). Furthermore, an analysis of the time spent by mice engaging in various activities within the cage indicates that MEAI-treated mice exhibit a preference for voluntary activities such as wheel running, pedestrian locomotion, and extended interaction times with food and water dispensers (FIG. 6G).

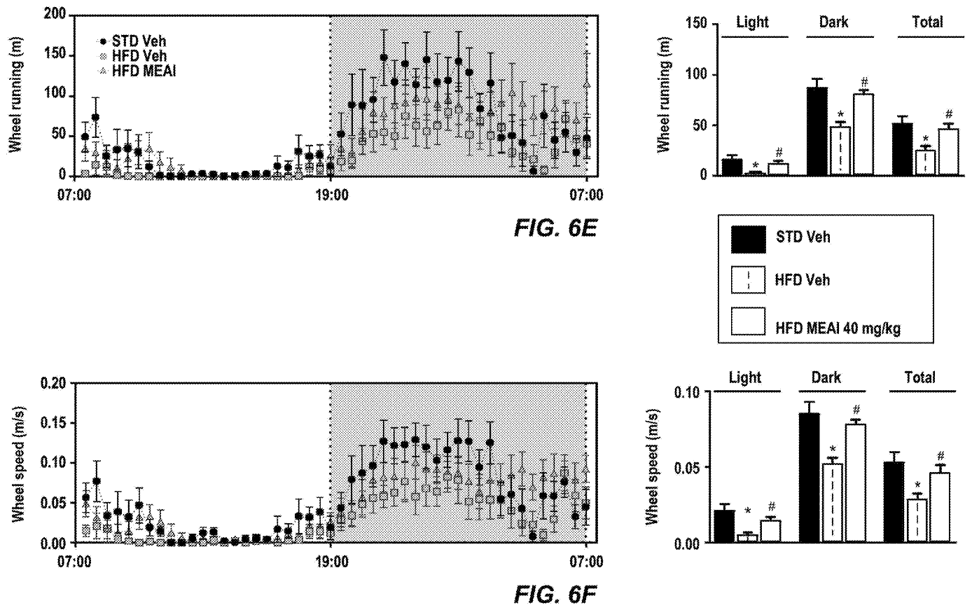

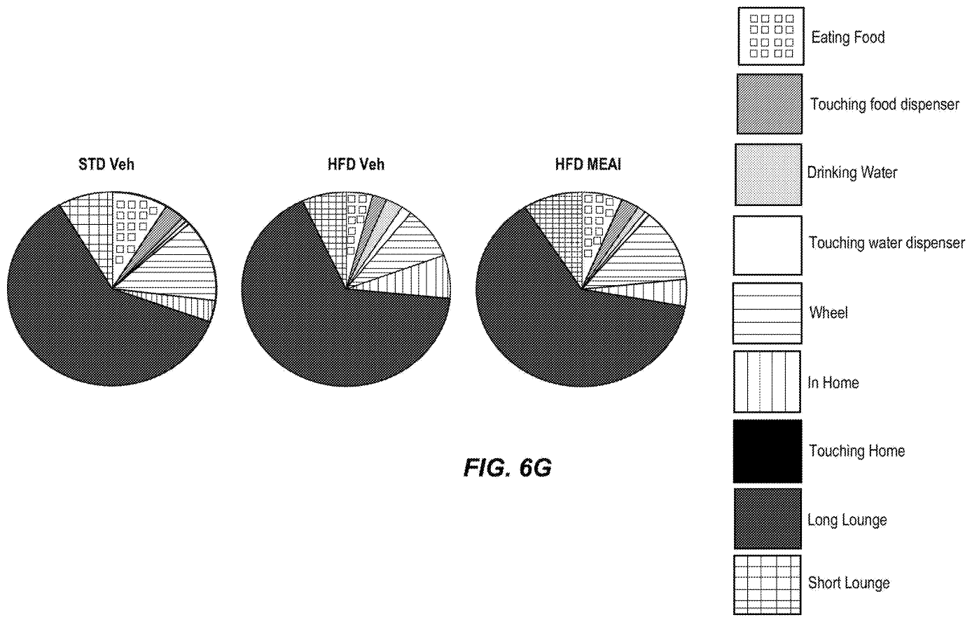

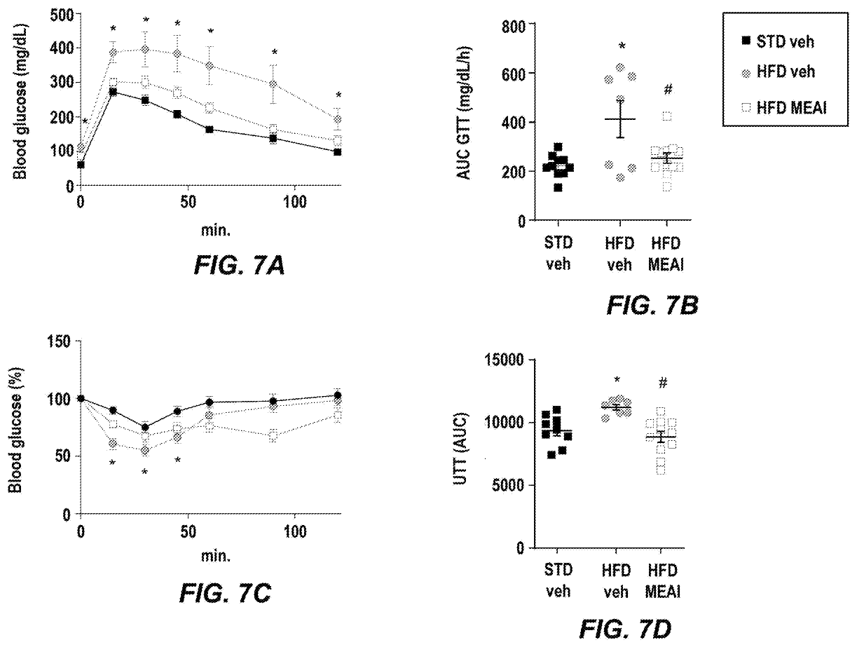

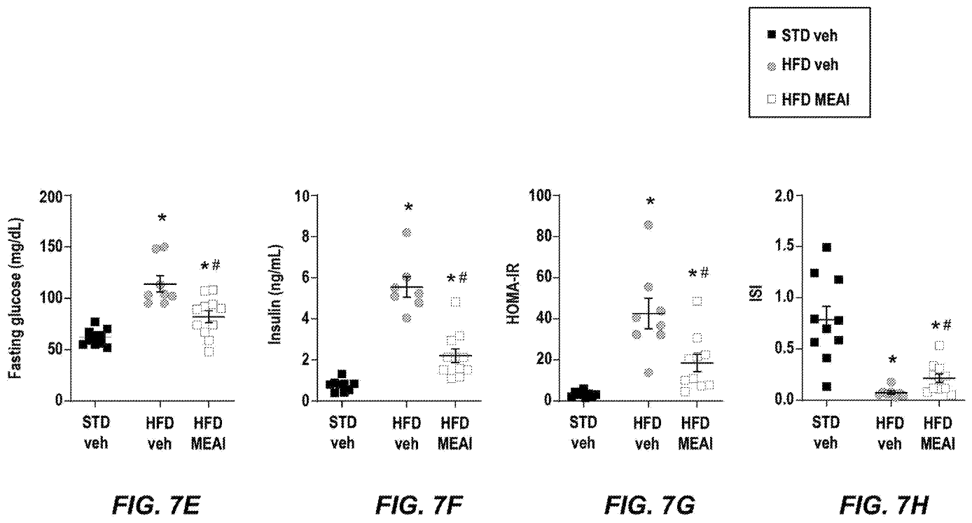

MEAI Improves Glycemic Control in DIO Mice. Obesity is a well-known contributor to insulin resistance and hyperglycemia, which can ultimately lead to the onset of diabetes. In the DIO model, a substantial impairment in glucose tolerance and an increase in hyperinsulinemia was observed, as demonstrated by the results of glucose and insulin tolerance tests. However, following treatment with MEAI, a significant improvement in glucose metabolism was observed (FIG. 7A-D), with fasting blood glucose and insulin levels also being reduced (FIG. 7E, F). These beneficial effects of MEAI were reflected in HOMA-IR and ISI (FIG. 7G, H), suggesting that MEAI has a positive impact on glucose metabolism. Additionally, MEAI normalized insulin sensitivity (FIGS. 7C, and D), indicating positive effects on glucose metabolism.

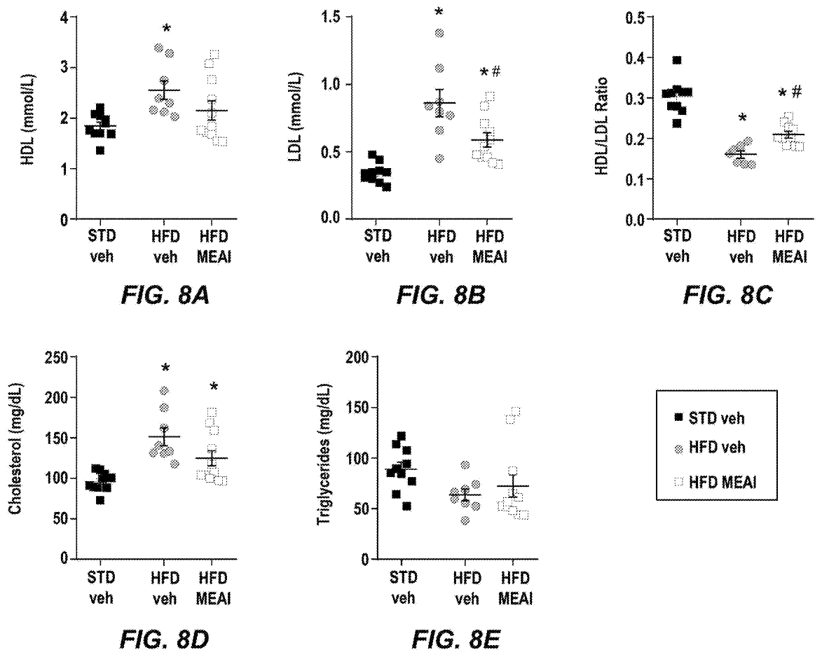

Treatment with MEAI ameliorates HFD-induced dyslipidemia. To investigate whether MEAI can alleviate the dyslipidemia commonly associated with obesity, the blood lipid profile was analyzed. The results showed that treatment with MEAI significantly reduced the levels of LDL compared to the HFD vehicle group, without notable changes in HDL levels. This reduction in LDL levels was accompanied by an increased HDL-to-LDL ratio, indicating a positive effect on lipid metabolism (FIG. 8A-C). Furthermore, there was a tendency towards decreased cholesterol levels in the MEAI-treated group (FIG. 8D), although this change was not statistically significant. However, no significant alterations in circulating triglyceride levels was observed in any of the study groups (FIG. 8E). These findings suggest that MEAI may have a potential therapeutic effect on dyslipidemia associated with obesity.

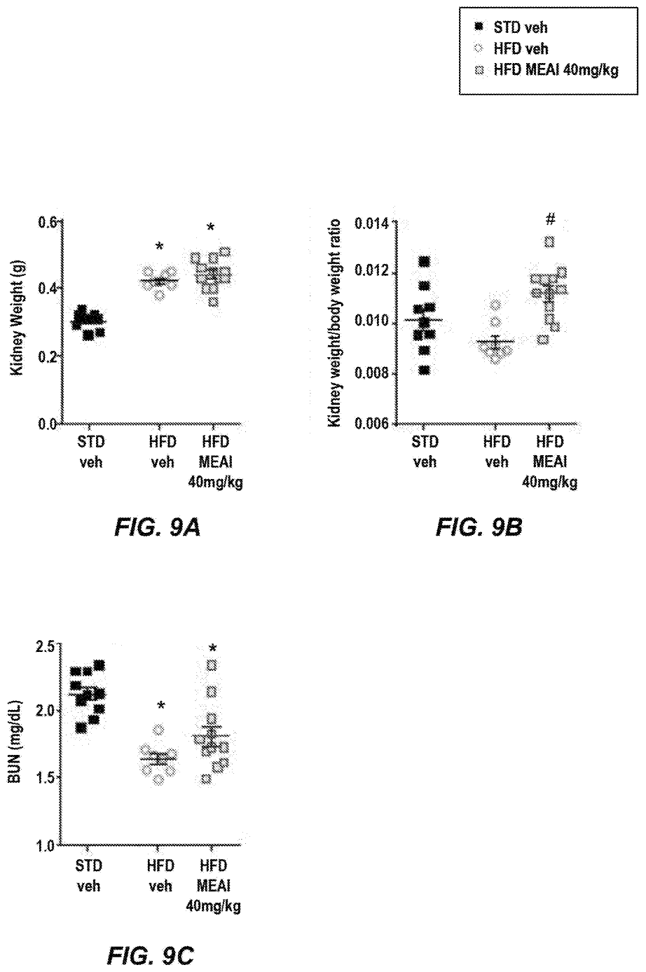

Initial testing to check the effect of MEAI on renal function show that kidney-to-body weight ratio is normalized following MEAI administration, accompanied by slight improvements of blood urea nitrogen (BUN) levels (FIGS. 9A-C).

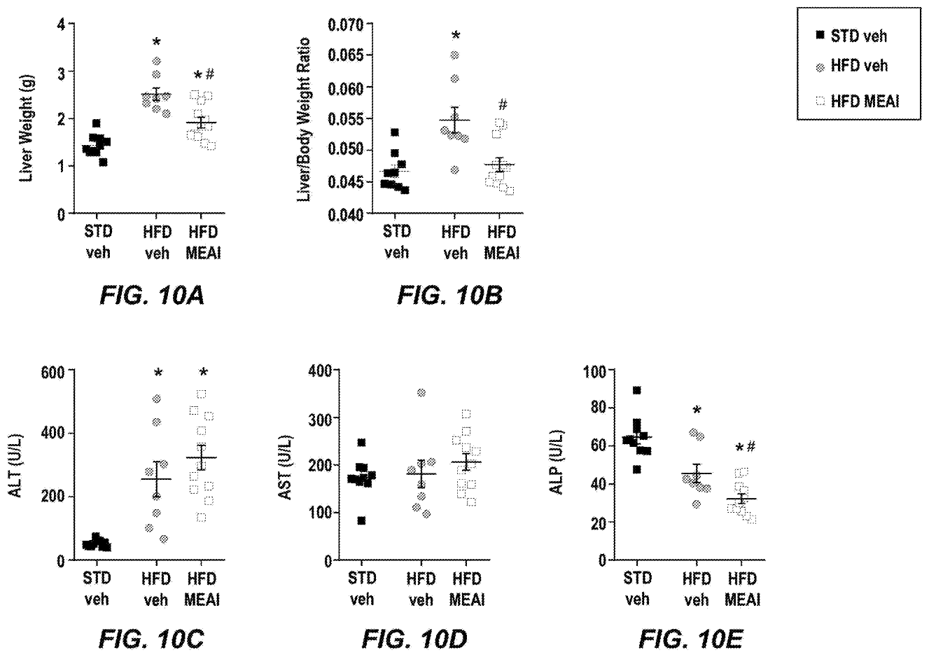

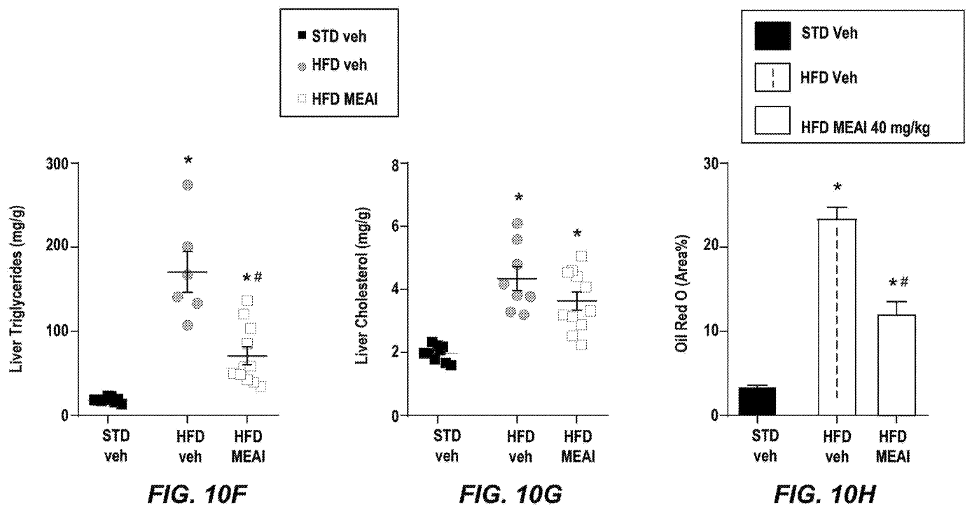

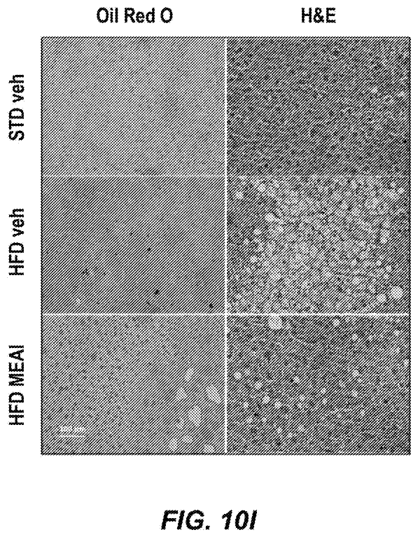

MEAI Reversed Obesity-Induced Hepatic Dysfunction and Steatosis. Obesity is a well-established risk factor for the development of NAFLD, characterized by hepatic steatosis resulting from an imbalance between hepatic fatty acid uptake, synthesis, oxidation, and export. Given the promising effects of MEAI on body weight, fat oxidation, and circulating lipid levels, its impact on liver steatosis was investigated. The findings demonstrate that MEAI treatment reduced liver weight (FIG. 10A) and normalized its ratio to body weight (FIG. 10B) in HFD-fed mice. Although MEAI administration did not significantly alter ALT or AST levels compared to the HFD-vehicle group, it significantly reduced ALP levels, which may suggest reduced liver injury (FIG. 10C-E). Moreover, treatment with MEAI had a positive effect on hepatic lipid accumulation, as evidenced by the significant reductions in liver triglycerides compared to the HFD vehicle control and a trend toward a reduction in hepatic cholesterol levels (FIG. 10F, G). These findings were further supported by a decrease in Oil Red O staining and reduced lipid vacuole numbers in MEAI-treated livers compared to HFD vehicle-treated group (FIG. 10H, I). Overall, these findings suggest that MEAI may have a beneficial effect on hepatic lipid accumulation and liver function in the context of obesity-associated NAFLD.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description or examples.

We claim:

1. A method for treating a metabolic condition in a subject in need thereof, comprising administering to the subject a therapeutically acceptable amount of a pharmaceutical composition comprising 5-methoxy-2-aminoindan or a pharmaceutically acceptable salt thereof, wherein the treatment reduces one or more metabolic syndromes in the subject.

2. The method according to claim 1, wherein the 5-methoxy-2-aminoindan or pharmaceutically acceptable salt thereof is administered as a dose of about 20 to about 520 mg.

3. The method according to claim 1, wherein the 5-methoxy-2-aminoindan or pharmaceutically acceptable salt thereof is administered as a dose of about 0.5 to about 40 mg.

4. The method according to claim 1, wherein the 5-methoxy-2-aminoindan or pharmaceutically acceptable salt thereof is administered as a dose of about 20 to about 100 mg, about 25 to about 90 mg, about 30 to about 80 mg, about 40 to about 70 mg, or about 50 to about 60 mg.

5. The method according to claim 2, wherein the dose is administered in a single dose or as more than one divided dose.

6. The method according to claim 2, wherein the dose is administered daily in a single dose or as more than one divided dose.

7. The method according to claim 2, wherein the 5-methoxy-2-aminoindan or a pharmaceutically acceptable salt thereof is administered twice a day.

8. The method according to claim 1, wherein the therapeutically effective amount comprises about 0.0084 to about 0.67 mg/kg body weight/day, about 0.33 to about 8.67 mg/kg body weight/day, about 0.33 to about 1.67 mg/kg body weight/day, about 0.42 to about 1.5 mg/kg body weight/day, about 0, to about 1.33 mg/kg body weight/day, about 0.67 to about 1.17 mg/kg body weight/day, or about 0.83 to about 1.0 mg/kg body weight/day.

9. The method according to claim 1, wherein the pharmaceutical composition further comprises at least one pharmaceutically acceptable carrier and/or excipient.

10. The method according to claim 9, wherein the pharmaceutical composition is a free-flowing powder, a tablet, a capsule, a lozenge, a liquid, a liquid concentrate, suspension, or a syrup.

11. The method according to claim 10, wherein the pharmaceutical composition is a unit dosage form composition.

12. The method according to claim 11, wherein an amount of 5-methoxy-2-aminoindan or pharmaceutically acceptable salt thereof in the unit dosage form is about 20 to about 520 mg, about 0.5 to about 40 mg, about 20 to about 100 mg, about 25 to about 90 mg, about 30 to about 80 mg, about 40 to about 70 mg, or about 50 to about 60 mg.

13. The method according to claim 12, wherein the amount of 5-methoxy-2-aminoindan or pharmaceutically acceptable salt thereof is about 50 mg.

14. The method according to claim 1, wherein administration of the pharmaceutical composition is oral, sublingual, buccal, vaginal, rectal, parenteral, transdermal, or by inhalation.

15. The method of claim 14, wherein the parenteral administration is intravenous, intramuscular, or subcutaneous.

16. A method for treating a metabolic condition in a subject in need thereof, comprising administering to the subject a therapeutically acceptable amount of a pharmaceutical composition comprising 5-methoxy-2-aminoindan or a pharmaceutically acceptable salt thereof, and a pharmaceutical composition comprising an N-acylethanolamine or a pharmaceutically acceptable salt thereof, wherein the treatment reduces one or more metabolic syndromes in the subject.

17. The method according to claim 16, wherein the 5-methoxy-2-aminoindan or pharmaceutically acceptable salt thereof is administered as a dose of about 20 to about 520 mg.

18. The method according to claim 16, wherein the 5-methoxy-2-aminoindan or pharmaceutically acceptable salt thereof is administered as a dose of about 0.5 to about 40 mg.

19. The method according to claim 16, wherein the 5-methoxy-2-aminoindan or pharmaceutically acceptable salt thereof is administered as a dose of about 20 to about 100 mg, about 25 to about 90 mg, about 30 to about 80 mg, about 40 to about 70 mg, or about 50 to about 60 mg.

20. The method according to claim 17, wherein the dose is administered in a single dose or in more than one divided dose.

21. The method according to claim 17, wherein the dose is administered daily in a single dose or in more than one divided dose.

22. The method according to claim 19, wherein the 5-methoxy-2-aminoindan or a pharmaceutically acceptable salt thereof is administered twice a day.

23. The method according to claim 16, wherein the therapeutically effective amount of the 5-methoxy-2-aminoindan or pharmaceutically acceptable salt thereof comprises about 0.0084 to about 0.67 mg/kg body weight/day, about 0.33 to about 8.67 mg/kg body weight/day, about 0.33 to about 1.67 mg/kg body weight/day, about 0.42 to about 1.5 mg/kg body weight/day, about 0, to about 1.33 mg/kg body weight/day, about 0.67 to about 1.17 mg/kg body weight/day, or about 0.83 to about 1.0 mg/kg body weight/day.

24. The method according to claim 16, wherein the pharmaceutical composition further comprises at least one pharmaceutically acceptable carrier and/or excipient.

25. The method according to claim 24, wherein the pharmaceutical composition is a free-flowing powder, a tablet, a capsule, a lozenge, a liquid, a liquid concentrate, suspension, or a syrup.

26. The method according to claim 25, wherein the pharmaceutical composition is a unit dosage form composition.

27. The method according to claim 26, wherein an amount of 5-methoxy-2-aminoindan or pharmaceutically acceptable salt thereof in the unit dosage form is about 20 to about 520 mg, about 0.5 to about 40 mg, about 20 to about 100 mg, about 25 to about 90 mg, about 30 to about 80 mg, about 40 to about 70 mg, or about 50 to about 60 mg.

28. The method according to claim 27, wherein the amount of 5-methoxy-2-aminoindan or pharmaceutically acceptable salt thereof is about 50 mg.

29. The method according to claim 16, wherein administration of the pharmaceutical composition is oral, sublingual, buccal, vaginal, rectal, parenteral, transdermal, or by inhalation.

30. The method of claim 29, wherein the parenteral administration is intravenous, intramuscular, or subcutaneous.

31. The method of any one of claims 16 to 30, wherein the N-acylethanolamine is selected from the group consisting of N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), salts thereof and any combination thereof.

32. The method according to claim 31, wherein the N-acylethanolamine is palmitoylethanolamide or a pharmaceutically acceptable salt thereof.

33. The method of any one of claims 16 to 32, wherein the N-acylethanolamine or pharmaceutically acceptable salt thereof is administered as a dose of about 200 to about 1800 mg, about 250 to about 1550 mg, about 300 to about 1200 mg, about 350 to about 950 mg, about 400 to about 700 mg, about 450 to about 600 mg, or about 500 to about 550 mg.

34. The method according to claim 33, wherein the dose is administered in a single dose or in more than one divided dose.

35. The method according to claim 33, wherein the dose is administered daily in a single dose or in more than one divided dose.

36. The method according to claim 35, wherein the N-acylethanolamine or a pharmaceutically acceptable salt thereof is administered twice a day.

37. The method according to claim 16, wherein the therapeutically effective amount of the N-acylethanolamine or pharmaceutically acceptable salt thereof comprises about 2.5 to about 36.0 mg/kg body weight/day, about 3.12 to about 31.0 mg/kg body weight/day, about 3.75 to about 24.0 mg/kg body weight/day, about 4.38 to about 19.0 mg/kg body weight/day, about 5.0 to about 14.0 mg/kg body weight/day, about 5.62 to about 12.0 mg/kg body weight/day, or about 6.25 to about 11.0 mg/kg body weight/day.

38. The method of any one of claims 16 to 37, wherein the N-acylethanolamine is administered simultaneously with the 5-methoxy-2-aminoindan.

39. The method of claim 38, wherein the N-acylethanolamine and the 5-methoxy-2-aminoindan are administered in a single pharmaceutical composition.

40. The method of claim 1 or 16, wherein administration is oral, mucosal, nasal, sublingual, inhalational, topical, rectal, vaginal, or parenteral route.

41. The method of claim 40, wherein the parenteral administration is intravenous, intramuscular, or subcutaneous.

42. The method of any one of claims 1 to 41, wherein treating metabolic syndrome involves one or more of decreasing blood pressure, decreasing blood sugar, reducing body fat around the waist, normalizing abnormal cholesterol or triglyceride levels, reducing obesity, reducing overweight, reducing body weight, increasing lean mass, reducing fat mass, reducing adiposity, increasing energy expenditure, improving glycemic control, decreasing hepatic steatosis, decreasing sugar intake, decreasing food intake, maintaining glucose homeostasis, lowering dyslipidemia, or preserving liver function.

43. The method of claim 42, wherein improving glycemic control involves one or more of improving glucose metabolism, reducing fasting blood glucose level, or reducing insulin level.

44. The method of claim 42, wherein increasing energy expenditure involves one or more of increasing oxygen consumption and carbon dioxide emission, increasing fat oxidation, or increasing locomotive activity.

45. The method of claim 42, wherein treating metabolic syndrome reduces obesity.

46. The method of claim 45, wherein treating metabolic syndrome involves reducing overweight associated with obesity.

47. The method of claim 45 or 46, wherein treating metabolic syndrome preserves lean mass of the subject.

48. The method of claim 45 or 46, wherein treating metabolic syndrome decreases fat mass of the subject.

49. The method of claim 45 or 46, wherein treating metabolic syndrome reduces adiposity of the subject.

50. The method of claim 45 or claim 46, wherein treating metabolic syndrome increases energy expenditure.

51. The method of claim 50, wherein food consumption is unchanged.

52. The method of claim 59, wherein fat utilization is increased.

53. The method of claim 50, wherein locomotive activity is normalized without over-stimulatory effects.

54. The method of claim 45 or claim 46, wherein treating metabolic syndrome improves glycemic control.

55. The method of claim 45 or claim 46, wherein treating metabolic syndrome reverses hyperglycemia, glucose intolerance, or hyperinsulinemia.

56. The method of claim 45 or claim 46, wherein treating metabolic syndrome improves hepatic steatosis.

57. The method of claim 56, wherein improving hepatic steatosis involves one or more of reducing hepatic lipid accumulation, hepatic triglyceride levels, or hepatic cholesterol levels.

58. The method of claim 45 or claim 46, wherein treating metabolic syndrome preserves glucose homeostasis.

59. The method of claim 58, wherein preserving glucose homeostasis involves one or more of enhancing glucose tolerance, attenuating insulin resistance, reducing dyslipidemia, or reducing hepatic lipid accumulation.

60. Use of a pharmaceutical composition comprising 5-methoxy-2-aminoindan or a pharmaceutically acceptable salt thereof for treating a metabolic condition as in any one of claims 1-59.

61. Use of a pharmaceutical composition comprising 5-methoxy-2-aminoindan or a pharmaceutically acceptable salt thereof, and an N-acylethanolamine (e.g., palmitoylethanolamide) or a pharmaceutically acceptable salt thereof, for treating a metabolic condition as in any one of claims 16-59.
